(19) United States
(12) Patent Application Publication
(10) Pub. No.: US 2017/0304367 A1
(43) Pub. Date: Oct. 26, 2017

(54) METHODS OF TREATING HYPOXIA-ASSOCIATED OPTICAL CONDITIONS WITH CARTILAGE OLIGO MATRIX PROTEIN-ANGIOPOIETIN 1 (COMP-ANG1)

(71) Applicant: University of Utah Research Foundation, Salt Lake City, UT (US)

(72) Inventors: Balamurali K. Ambati, Sandy, UT (US); Hironori Uehara, Salt Lake City, UT (US); Judd Hoon, Salt Lake City, UT (US)

(73) Assignee: University of Utah Research Foundation, Salt Lake City, UT (US)

(21) Appl. No.: 15/523,639

(22) PCT Filed: Oct. 29, 2015
(86) PCT No.: PCT/US2015/058159

§ 371 Date: May 1, 2017

Related U.S. Application Data
(60) Provisional application No. 62/072,349, filed on Oct. 29, 2014.

(51) Int. Cl. A61K 35/28; A61K 47/34; A61K 38/18; A61K 47/42; A61K 9/00; A61K 47/00; A61K 38/00

(52) U.S. Cl. CPC A61K 35/28; A61K 47/42; A61K 47/34; A61K 9/0048; A61K 38/1891; A61K 38/00; A61K 47/00

(57) ABSTRACT

Methods, compositions, and systems for treating ischemia-associated and other ocular conditions using cartilage oligo matrix protein-Angiopoietin 1 (COMP-Ang1) are disclosed and described.

### GOVERNMENT INTEREST

This invention was made with government support under Grant number 5T32DK091317 awarded by National Institutes of Health. The government has certain rights in the invention.

### BACKGROUND

Various ocular conditions affect the retina of the eye and consequently impinge the visual health and stability of those suffering from such conditions. Retinal conditions can include glaucoma, central retinal artery occlusion, retinal detachment, macular holes, diabetic retinopathy, diabetic macular edema, and many more. As a specific example, diabetic retinopathy (DR) affects nearly 30% of people with diabetes and is the leading cause of blindness in the working-age population. The treatments for DR fail to improve vision in a significant number of patients, and have risks of retinal burns, detachment, hemorrhage, infection, or pain. In fact, current treatment of DR only slows, but does not stop, disease progression. Furthermore, some conditions, such as central retinal artery occlusion, currently don't have any method of treatment, resulting in either partial or complete loss of vision without hope of improvement.

### BRIEF DESCRIPTION OF THE DRAWINGS

FIG. 1 represents a genetic map used in one embodiment of an expression vector.

FIGS. 2A-2D illustrate a schematic of a treatment paradigm and plasmid design used in some of the Examples. (A) Ins2Akita mice were treated at 2 months of age with intravitreal injection of either PBS or AAV2 expressing either AcGFP (control) or COMP-Ang1 (treatment). Retinas were harvested at 6 months of age (4 months after injection). (B) In a second cohort of mice, mice were initially injected at the delayed age of 6 months, followed two weeks later by ECFC injection and six weeks later by retinal harvest. (C,D) An embodiment of a plasmid design for COMP-Ang1 and AcGFP used in some of the examples.

FIGS. 3A-3B illustrate experimental results indicating that intravitreal injection of a COMP-Ang1 agent does not cause damage in wild type (WT) retina. (A) WT mice were given intravitreal injections of PBS or AAV2 expressing either AcGFP or COMP-Ang1 and assessed for retinal function via ERG. Compared to PBS injected retinas, there was no difference in b-wave amplitude between groups. (B) These same mice were also tested for visual tracking response over 5 weeks. Time 0 represents the week before injection. There was no difference between groups.

FIGS. 4A-4C illustrate experimental results indicating that AAV2.COMP-Ang1 prevents diabetes-induced neural dysfunction. (A) Representative example of ERG response from all groups of mice. Electrical retinal response was elicited and the amplitude of b-wave during scotopic conditions at −3.62 log (Cd s/m²) (−40 dB), −2.62 log (Cd s/m²)(−30 dB), −1.62 log (Cd s/m²) (−20 dB), intensity was recorded. (B) Decreased amplitudes were recorded in Ins2Akita mice treated with PBS or AAV2.AcGFP compared to WT mice and AAV2.COMP-Ang1 prevented the decrease in amplitude. *p<0.01, ANOVA, #p<0.01 compared to AAv2.AcGFP. Assessing visual acuity was accomplished by testing optomotor tracking response of Ins2Akita mice treated with AAV2.COMP-Ang1 or control compared to WT. (C) Ins2Akita mice exhibited decreased optokinetic tracking response (units=cycles/degree). AAV2.COMP-Ang1 prevented the decrease in visual response; at least six mice from each group were tested, data are mean±stdev. *p<0.01, ANOVA. Post-hoc comparisons with a Tukey test to compare means of each group.

FIGS. 5A-5H illustrate experimental results indicating that intravitreal injection of AAV2 successfully infects the inner retina. (A) AAV2.AcGFP expression was followed with in vivo confocal ophthalmoscopy (Heidelberg Spectralis) as early as 1 week after injection. Expression remained evident beyond 4 months post-injection. (B) Ex vivo retinal flat mount demonstrating AAV2.AcGFP expression in all four retinal quadrants. (C) Retinal cross-sections demonstrating AAV2.AcGFP expression primarily located in GCL-IPL layer of the retina. (D) Semi-quantitative RT-PCR demonstrated that COMP-Ang1 was expressed only in the eyes of mice injected with AAV2.COMP-Ang1. Flag protein was also found only in mice treated with the AAV2.COMP-Ang1 (E). To localize COMP-Ang1 transcript within the retina, in situ hybridization was utilized. (F) Expression of Ang1 was found in the retinas of mice treated with AAV2.AcGFP. (G) Mice treated with AAV2.COMP-Ang1 showed a demonstrable increase in Ang1 (detecting both native Ang1 and COMP-Ang1 transgene) primarily located in the GCL-IPL. (H) Negative control with Ang1 sense probe.

FIGS. 6A-6F illustrate experimental results indicating that AAV2.COMP-Ang1 mitigates diabetic retinal capillary dropout (A) Representative retinal flatmounts prepared from six month-old mice and stained for isolectin (endothelial cell marker, green) and α-SMA (smooth muscle marker, red). (B) Magnified view of retina stained with isolectin and NG2 (pericyte marker). Ins2Akita mice experienced pericyte and endothelial dropout; the latter was prevented with a single intravitreal dose of AAV2.COMP-Ang1. (C) Trypsin digest featuring retinas representative of each group. Black arrowheads denote acellular capillaries. (D) Quantification using ImageJ of endothelial coverage and (E) pericyte coverage. (F) Acellular capillaries were manually counted and averaged over an area 1 mm². Eight eyes were used in each analysis, data are mean±stdev. *p<0.01, ANOVA. Post hoc comparisons with a Tukey test to compare means of each group. Scale bars=600 μm (A), 100 μm (B), 200 μm (C).

FIG. 7A includes a representative graph of electrical cell substrate impedance sensing (ECIS) of human retinal microvascular endothelial cells (HrMVECs) with COMP-Ang1 (100 ng/mL), VEGF (50 ng/mL), or control (PBS) added to the media. COMP-Ang1 increased resistance of HrMVECs (n=3).

FIG. 7B shows experimental results indicating that Evans Blue extravasation from the retina of Ins2Akita mouse was increased compared to control; treatment with AAV2.COMP-Ang1 returned vascular hyperpermeabilty to control levels. Eyes from eight mice were used in each analysis; data are mean±stdev. *p<0.01 compared to WT, *p=0.02 compared to AAV2.AcGFP.

FIG. 7C includes example results where Fluorescein angiography (FA) did reveal any leakage in diabetic mice; however, utilizing GFP or the NIR fluorophore ZW800 conjugated to aminated latex microspheres (GFP-ms, or ZW800-ms; 100 nm in diameter) in vivo leakage was captured using the FA or ICG imaging modality on Spectralis, respectively. Note that background GFP fluorescence of the AAV2.AcGFP treated diabetic mice masked signal from the GFP-microspheres.

FIG. 8A-8F show increases in endothelial resistance were correlated with decreased Src phosphorylation (A) and increased VE-cadherin (B) in HrMVECs as demonstrated by Western blot (n=3). Western blot from Ins2Akita mouse retinas demonstrating decreased VEGF-A (C) and increased VE-cadherin (D) in mice treated with AAV2.COMP-Ang1. (E) Similar to Ins2Akita mice, intravitreal injection of AAV2.COMP-Ang1 increased VE-Cadherin and decreased VEGF in the retinas of WT mice when compared to AAV2.AcGFP. (F) COMP-Ang1 increased Akt phosphorylation at the serine 473 residue in both ECFCs and HUVECs.

FIG. 9A-D includes experimental data indicating that COMP-Ang1 enhances vascular barrier function and reduces retinal hypoxia in the diabetic retina. (A) COMP-Ang1 reduced TNF-α induced leukocyte rolling in cultured HrMVECs (n=6 per group). *p<0.01, ANOVA. (B) Diabetes induced leukocyte rolling in the retinal vasculature, as captured by acridine orange leukocyte fluorography AOLF. (C) Representative image of AOLF with white arrowheads pointing to adherent and rolling leukocytes. COMP-Ang1 prevents leukostasis and inflammation in this model of diabetic retinopathy. (D) Representative retinas (four mice per group) from mice treated with hypoxyprobe (pimonidazole). COMP-Ang1 reduced hypoxia in diabetic mouse retinas. Scale bars: 600 μm (D), *p<0.01, ANOVA. Post hoc comparisons with a Tukey test to compare means of each group.

FIGS. 10A-10F includes example results indicating that AAV2.COMP-Ang1 prevents diabetes-induced GC-IPL degeneration. (A) Representative figures from optical coherence tomography (OCT) measuring retinal thickness. The red line, generated by OCT software, indicates the retinal surface and Bruch's membrane. Scale bars=200 (B) Cross sections of six month-old retinas from WT, or Ins2Akita mice treated with PBS, AAV2.AcGFP, or AAV2.COMP-Ang1 stained with DAPI. (C) View of the GC-IPL from mice stained for VE-cadherin (red) or nuclei (DAPI, blue) demonstrating increased VE-cadherin and nuclear staining. Scale bars 30 μm (right). (D) Quantification of retinal thickness from OCT showing that AAV2.COMP-Ang1 prevented diabetes-induced retinal thinning as measured in vivo (*p<0.01 vs. both WT and AAV2.AcGFP). AAV2.COMP-Ang1 prevented diabetes-induced inner retinal layer loss (*p=0.03 ANOVA, with post hoc Tukey test) as measured by nuclei counted in the GC-IPL in retinal cross sections. (E) Representative retinal flat mounts stained with BRN3 (red; a marker for retinal ganglion cells) and isolectin (green; marker for vessels) and DAPI (blue). Qualitatively, fewer peripheral RGCs are observed in PBS-treated Akita retina. (F) Ins2Akita mice showed no difference in central RGCs with either treatment but there was a trend towards reduced peripheral RGCs in PBS-treated mice vs. COMP-Ang1 treated mice (17% fewer ganglion cells in PBS group (p=0.07)). At least six eyes from each group were tested, data are mean±stdev. *p<0.01, ANOVA. Post hoc comparisons with a Tukey test to compare means of each group. p, peripheral; c, central.

FIGS. 11A-11D includes example results indicating that AAV2.COMP-Ang1 enhances ECFC engraftment into the diabetic retina and prevents further visual decline. (A) Endothelial colony-forming cells (ECFCs) were plated on collagen-coated wells and assayed for migration potential under increasing doses of COMP-Ang1. (B) Additionally, 3D tube formation was tested in matrigel. COMP-Ang1 increased migration and tube formation in a dose dependent manner with maximal effects exerted at 500 ng/mL. Qdot-655 labeled ECFCs were injected intravitreally into aged diabetic mice (6 months, arrow in D) after the mice had been treated with COMP-Ang1 or control. Three days later retinas were harvested and stained for blood vessels (isolectin 546) and flatmounted for confocal analysis. COMP-Ang1 increased ECFC integration into the diabetic retinal vasculature. (D) Mice treated with COMP-Ang1 or control plus ECFCs were analyzed for visual tracking ability. COMP-Ang1 plus ECFCs prevented further declines in spatial frequency threshold. *p<0.001 in vitro experiments were performed in triplicate on three different ECFC clones (total of nine experiments per condition). In vivo experiments were performed on five mice per group (ten eyes). Scale bars (C): 600 μm (top), 150 μm (middle), and 90 μm (bottom). *p<0.01, ANOVA. Post hoc comparisons with a Tukey test to compare means of each group.

FIG. 12A includes some example Fluorescein angiography results performed before and after induced retinal artery occlusion.

FIG. 12B includes some example retinal cross-sections from untreated and treated mice.

FIGS. 12C-12D include example retinal cross-sections from mice that are stained for various markers.

FIGS. 13A-13D include example retinal cross-sections from mice that are stained for various markers, including PCNA, MCM6, and SOX2.

FIG. 14 includes example comparative results for visual tracking in treated mice after induced CRAO.

FIGS. 15A-15C include example retinal cross-sections from mice that are stained for various markers, including PCNA and NeuN.

FIGS. 16A-16B include example retinal cross-sections from mice that are stained for various markers, including PCNA and TuJ1.

FIGS. 17A-17B include example retinal cross-sections from mice that are stained for various markers, including Brn3 and TuJ1.

FIGS. 18A-18B include example retinal cross-sections from mice that are stained for various markers, including PCNA and SOX2.

### DESCRIPTION OF EMBODIMENTS

Although the following detailed description contains many specifics for the purpose of illustration, a person of ordinary skill in the art will appreciate that many variations and alterations to the following details can be made and are considered to be included herein. Accordingly, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used herein have the same meaning as commonly understood by one of ordinary skill in the art to which this disclosure belongs.

As used in this specification and the appended claims, the singular forms “a,” “an” and “the” include plural referents unless the context clearly dictates otherwise. Thus, for example, reference to “a cell” includes a plurality of such cells.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term, like “comprising” or “including,” it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, a “cartilage oligo matrix protein-Angiopoietin1 agent” refers to a chemical or biological agent that includes cartilage oligo matrix protein-Angiopoietin1 (COMP-Ang1) protein or a precursor or homologue thereof. It also refers to an expression vector or any other mechanism of stimulating or inducing endogenous production of COMP-Ang1 and related molecules.

As used herein, a “subject” refers to an animal. In one aspect the animal may be a mammal. In another aspect, the mammal may be a human.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this specification support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of invention embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Ocular conditions that affect the retina of the eye can result in partial or permanent loss of vision in a subject. Such conditions can include glaucoma, central retinal artery occlusion, retinal detachment, macular holes, diabetic retinopathy, diabetic macular edema, and others. As one example, central retinal artery occlusion (CRAO) results when blood flow through the central retinal artery is blocked. This blockage causes a depletion of vital oxygen and nutrients necessary for neuron survival and development in retinal tissue. The outcome is either partial or complete loss of vision that can be largely irreversible if not treated within about 90 minutes of the blockage.

As another example, diabetic retinopathy (DR) affects nearly 30% of people with diabetes and is the leading cause of blindness in the working-age population. Among chronic eye diseases, DR has the highest annual spending per patient and imposes tremendous economic and quality-of-life burdens due to the young median age at diagnosis.

The leading cause of vision loss in DR is diabetic macular edema (DME), largely induced by vascular hyperpermeability associated with breakdown of the blood retinal barrier (BRB). Thus, DME is a condition in which fluid accumulates underneath the central macula due to a breakdown of the BRB. Current treatments for DME include laser photocoagulation, intravitreal agents that block vascular endothelial growth factor (VEGF), and/or intravitreal corticosteroids. Such treatments address the downstream consequences but not the vascular endothelial cell loss and ischemia underlying DME. Moreover, these therapies improve vision in only a minority of patients. Merely 23-33% of patients treated with ranibizumab and 34% of patients treated with aflibercept achieve significant visual gains. Because it creates small burns that can interfere with peripheral vision and overall visual performance, traditional treatment with laser photocoagulation is primarily employed to retard rather than reverse retinal non-perfusion. Intravitreal steroids have served as an alternative for patients who have contraindications or are resistant to anti-VEGF agents, but are inferior to VEGF inhibitors in recovering visual acuity and are associated with side effects like cataract and intraocular hypertension. Given the suboptimal outcomes, there is a need for a different approach to DME focusing on the reversal of retinal vascular damage and restoration of normal perfusion.

The underlying pathogenesis of DR is largely due to hyperglycemia. Hyperglycemia triggers an inflammatory response leading to leukocyte adhesion, microvascular occlusion, and consequent hypoxia. Further, hyperglycemia induces pericyte loss, compromising endothelial stability and blood retinal barrier (BRB) integrity. Eventual capillary degeneration, such as acellular capillaries, leads to retinal non-perfusion, exacerbating retinal hypoxia. Consequent pathological VEGF-induced angiogenesis is uncoordinated and results in immature, leaky vessels with inadequate perfusion, creating a vicious cycle of hypoxia-driven VEGF secretion and DME. Retinal ganglion cell (RGC) loss, neuronal dysfunction, and changes in vision are also seen in patients with DR, concurrently with vascular pathology. Thus, there is a need for a therapeutic agent capable of inducing vascular stabilization to promote normal perfusion of the metabolically demanding retinal neurons and thereby avert the sight-threatening sequelae of ischemia and herpermeability.

One such therapeutic agent is angiopoietin 1 (Ang 1), a vascular growth factor that has an abnormally low concentration in the vitreous of patients with DR. Ang1, via binding to the Tie2 endothelial receptor, fosters vessel quiescence & maturation, and suppresses vascular leakage by preventing VEGF-induced degradation of vascular endothelial (VE)-cadherin, a transmembrane protein in the adherens junction between endothelial cells that promotes vascular integrity and decreases vascular permeability. Further, Ang1 promotes survival of damaged vascular endothelial cells through the PI3K/Akt cascade. Thus, restoration of Ang1 signaling can serve as a potential treatment to prevent endothelial loss, retinal ischemia, and abnormal VEGF expression in DR. However, pharmaceutical development of Ang1 as a viable therapy has been hindered by its insolubility and aggregation. To overcome the limitations of native Ang1, a novel, stable, soluble and more potent version of Ang1, cartilage oligo matrix protein Ang1 (COMP-Ang1), has been developed.

COMP-Ang1 is an engineered, more stable, soluble, and potent version of naturally occurring Ang1, which induces vessel quiescence and maturation, and decreases vascular leakage by preventing VEGF-induced degradation of vascular endothelial (VE)-cadherin, a transmembrane protein in the adherens junction between endothelial cells that promotes vascular integrity and decreases vascular permeability. Further, Ang1 promotes survival of damaged vascular endothelial cells through PI3K/Akt signaling. However, the production of Ang1 can be adversely affected by aggregation and insolubility. Such aggregation and insolubility can be caused by disulfide linkages forming higher-order structures. COMP-Ang1 can be produced by modifying the N-terminus of the Ang1 protein to contain a short coiled-coil domain of cartilage oligomeric matrix protein, thus preventing the aggregation and insolubility problems associated with native Ang1 due to its modified structure, molecular weight, charge, and other factors.

Further, as previously discussed, acellular capillaries, which are a hallmark of diabetic retinopathy and lead to nonperfusion, can be recellularized and refunctionalized by endothelial progenitor cells (EPCs) or the like. However, it is noted that CD34+ EPCs, isolated from diabetic patients, have a decreased ability to associate with the existing vascular networks. The term EPC encapsulates a diverse group of cell-types with myeloid, haematopoietic or endothelial characteristics. A unique EPC sub-type, known as endothelial colony-forming cells (ECFCs, also known as outgrowth endothelial cells (OECs)), can encompass true endothelial progenitors, which fully integrate into blood vessels and serve to regenerate damaged vasculature in the ischemic retina, as well as other ischemic models.

Accordingly, embodiments are provided herein to address the concerns discussed above. One such embodiment includes a method of treating a disease or disorder of the eye. The method can include administering a therapeutically effective amount of a cartilage oligo matrix protein-Angiopoietin1 (COMP-Ang1) agent to an eye of a subject during a treatment period.

In one embodiment, the method can be a method of protecting, preserving and/or restoring the vasculature that provides oxygen and nutrients to the neurons in the retina. In one embodiment, the method can be a method of preventing neurovascular dysfunction in the eye. In one embodiment, the method can be a method of preserving, protecting and/or restoring neuronal function in retinal tissue following an ischemic or hypoxic event. In one embodiment, the method can be a method of preserving and/or protecting retinal neurons or neuronal function in retinal tissue due to an ischemic or hypoxic event. Accordingly, the current method can be used to treat a variety of ocular and systemic diseases and disorders. Non-limiting examples of the diseases or disorders that can be treated with the current method can include or be selected from the group consisting of neurovascular dysfunction, neuronal dysfunction, vascular hyperpermeability, retinal ischemia, retinal hypoxia, retinal hypoglycemia, retinal hyperglycemia, retinal stroke, central retinal artery occlusion (CRAO), central retinal vein occlusion, diabetic retinopathy, diabetic macular edema, pericyte dropout, endothelial cell dropout, capillary dropout, decreased blood-retinal barrier (BRB) integrity, leukocyte adhesion, inflammation, and the like. Capillary dropout can refer to a loss of normal capillaries due to diabetic pathogenesis.

In various, non-limiting aspects, treatments using the current technology can be directed toward retinal diseases or disorders. The retina is part of the central nervous system. Proper neural function in the retina is essential for transducing optical information to the brain. All retinal neurons can be adversely affected by ischemic and/or hypoxic events, which impair neural function in the retina. In one aspect, the method for preserving neuronal function in retinal tissue following an ischemic or hypoxic event can preserve neuronal function in all retinal neurons. In one aspect, neuronal function can be preserved in at least one of photoreceptors, bipolar cells, ganglion cells, horizontal cells, amacrine cells, and combinations thereof.

Retinal tissue can include all tissue or vasculature associated with the retina. Such tissue can include neural cells, supporting cells, vasculature associated with the retina, and all other cells associated with the retina. More specifically, retinal tissue can include at least one of the inner limiting membrane, the outer limiting membrane, the nerve fiber layer, the ganglion cell layer, the inner plexiform layer, the outer plexiform layer, the inner nuclear layer, the outer nuclear layer, the rod and cone layer, the pigment layer, any other tissues associated with the retina, any vasculature associated with the retina, and combinations thereof.

An ischemic event can include hypoxia, hypoglycemia, and depletion of other nutrients. Thus, an ischemic event can include any event where retinal tissue is inadequately supplied with oxygen, glucose and/or other nutrients. In one aspect, an ischemic event can occur due to inadequate vessel formation, resulting in leaky vessels and non-perfusion. In another aspect, an ischemic event can result from a blockage of any artery supplying oxygen, glucose, and/or other nutrients to the eye, such as, for example, a blockage of the central retinal artery. A hypoxic event or a hypoglycemic event can be similarly described. In a further aspect, a hypoxic event can occur due to any disease that causes depleted oxygen levels in the eye, such as, for example, anemia. In one aspect, a hypoxic event can result from poisoning, such as, for example, carbon monoxide poisoning. A hypoxic event can result from a variety of other circumstances known by those skilled in the art. Additionally, a hypoglycemic event can occur due to diabetes-related symptoms, a complication associated with medication used to treat diabetes, fasting, and other conditions known by those skilled in the art. Thus, the current technology can be used to treat ocular symptoms resulting from an ischemic, hypoxic, or hypoglycemic event.

In another embodiment, treatment can include reprogramming a Müller cell to function as a neural cell. Accordingly, a valuable embodiment of the current technology can include a method of reprogramming a Müller cell to function as a neural cell. In this embodiment, a therapeutically effective amount, or an activating or reprogramming amount, of a COMP-Ang1 agent can be administered to an eye of a subject to cause activation or reprogramming of Müller cells within the eye to function as a neural cell.

Müller cells act as support cells for retinal neurons. Although their unique shape can facilitate transmission of light through the retina, they merely direct optical information and do not perform the same sensory function as neurons. Without wishing to be bound by theory, and as described further in Example 9 below, it is believed that a COMP-Ang1 agent can cause these support cells to dedifferentiate to a progenitor phenotype. The progenitor cells subsequently differentiate into a functional neural cell in the retina to restore at least some neural function and visual capacity in the eye. Thus, this aspect or embodiment of the current technology can also help protect, preserve, and restore retinal neurons or neuronal function during or following an ischemic or hypoxic event.

In another embodiment, the method can also include administering a therapeutically effective amount of progenitor cells to an eye of the subject. A variety of suitable progenitor cells can be used. In one aspect, endothelial progenitor cells (EPCs), such as endothelial colony-forming cells (ECFCs), can be used. Progenitor or stem cells can be obtained from fresh or frozen umbilical cord blood, peripheral blood stem cells, bone-marrow derived stem cells, transformed, reprogrammed, or induced pluripotent stems cells, or any other suitable source.

In one aspect, a therapeutically effective amount of progenitor cells can include from about 5,000 cells to about 60,000,000 cells. In another aspect, the therapeutically effective amount of progenitor cells can be from about 10,000 to about 40,000,000 cells. In another aspect, the therapeutically effective amount of progenitor cells can be from about 20,000 to about 20,000,000 cells. In another aspect, the therapeutically effective amount can be from about 40,000 to about 10,000,000 cells. It is noted that the amounts and frequency of administration can depend on the patient, nature of the health condition, dosage of the progenitor or stem cells, and the like. Amounts and frequency of administration can be optimized according to these and other relevant considerations known in the art.

The progenitor or stem cells can be administered to the subject in any way suitable. In one aspect, the cells can be injected into the eye. In one specific aspect, the cells can be administered via intravitreal injection. In another aspect, administration can be systemic injection. The progenitor or stem cells can be administered as part of the same composition as the COMP-Ang1 agent or as a separate composition. The progenitor or stem cells can be administered as a composition that includes a suitable culture media, nutrient mixtures, or other sterile solution adapted to maintain the viability of the cells.

In some embodiments, the COMP-Ang1 agent can be administered jointly or concomitantly with the progenitor or stem cells, or it can be administered separately. In one aspect, the progenitor or stem cells are administered prior to the COMP-Ang1 agent. In one aspect, the progenitor or stem cells are administered after the COMP-Ang1 agent. In one aspect, the progenitor or stem cells are administered within about 30 minutes of one another. In one aspect, the progenitor or stem cells and the COMP-Ang1 agent are admixed and administered together in a single composition or formulation.

The COMP-Ang1 agent can be administered via a number of suitable methods. In one aspect, the COMP-Ang1 agent can be administered by at least one of injection, iontophoresis, eye drop, gel, ocular bandage or patch, ointment, and combinations thereof. In one specific aspect, a COMP-Ang1 agent can be administered via intravitreal injection. In another aspect, the COMP-Ang1 agent can be administered via systemic injection.

A COMP-Ang1 agent can include COMP-Ang1 protein, homologues of the COMP-Ang1 protein, modified COMP-Ang1 proteins that are equivalent in function, expression vectors adapted to express such proteins, and combinations thereof. In one aspect, the COMP-Ang1 agent can include COMP-Ang1 protein.

In another aspect, the COMP-Ang1 agent can include an expression vector or viral particle that is adapted to express COMP-Ang1, or a homologue thereof, within the eye. A variety of suitable expression vectors can be used. In one aspect, at least one of a lentivirus, an adenovirus, a cytomegalovirus, an adeno-associated virus (AAV), and combinations thereof can be used. In one aspect, the expression vector can be an AAV. The AAV can include or be selected from the group consisting of adeno-associated virus serotype 2 (AAV2), adeno-associated virus serotype 9 (AAV9), adeno-associated virus serotype 10 (AAV10), and combinations thereof. In one specific aspect, the expression vector can be an AAV2.

These expression vectors can be prepared using a variety of techniques. One such example of a method for preparing an expression vector will be illustrated using an AAV2. However, as will be recognized by one skilled in the art, the same or similar techniques can be used to prepare a COMP-Ang1 agent employing a different viral particle. In one example, plasmids of pAAV.COMP-Ang1 can be created by incorporating COMP-Ang1 cDNA from a pCMV-dhfr2-COMP-Ang1 into pAAV-MCS. An AAV viral vector can be converted to serotype 2. Cassettes from the plasmids can be integrated into the AAV2 vectors, driven by a CMV promoter, to generate AAV2.COMP-Ang1 or equivalent.

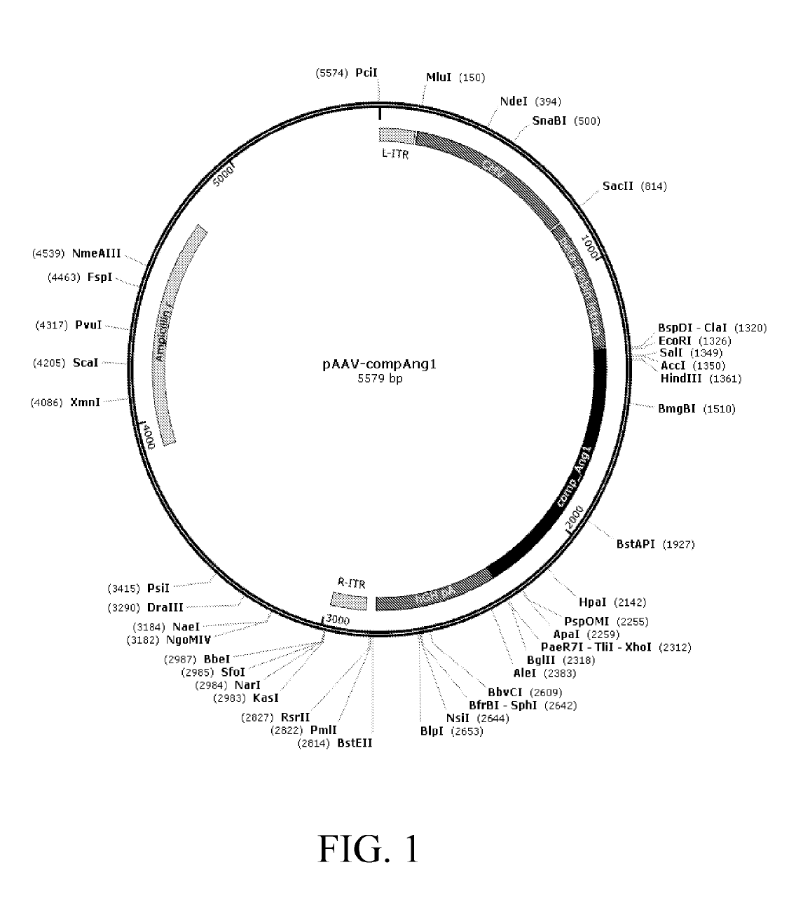

In one aspect, the AAV2.COMP-Ang1 expression vector can include the features illustrated in the genetic map shown in FIG. 1. COMP-Ang1 can be expressed in a number of target cell types within the eye. Target cell types can include retinal vascular endothelial cells, retinal ganglion cells, Muller cells, amacrine cells, bipolar cells, horizontal cells, microglial cells, astroglial cells, and infiltrating leukocytes. The COMP-Ang1 protein can be encoded by the following nucleotide sequence (SEQ ID 001):

atgaagacgatcatcgccctgagctacatcttctgcctggtattcgccga
ctacaaggacgatgatgacaaggggatcttagacctagccccacagatgc
ttcgagaactccaggagactaatgcggcgctgcaagacgtgagagagctc
ttgcgacagcaggtcaaggagatcaccttcctgaagaatacggtgatgga
atgtgacgcttgcggaggatcccttgtcaatctttgcactaaagaaggtg
ttttactaaagggaggaaaaagagaggaagagaaaccatttagagactgt
gcagatgtatatcaagctggttttaataaaagtggaatctacactattta
tattaataatatgccagaacccaaaaaggtgttttgcaatatggatgtca
atgggggaggttggactgtaatacaacatcgtgaagatggaagtctagat
ttccaaagaggctggaaggaatataaaatgggttttggaaatccctccgg
tgaatattggctggggaatgagtttatttttgccattaccagtcagaggc
agtacatgctaagaattgagttaatggactgggaagggaaccgagcctat
tcacagtatgacagattccacataggaaatgaaaagcaaaactataggtt
gtatttaaaaggtcacactgggacagcaggaaaacagagcagcctgatct
tacacggtgctgatttcagcactaaagatgctgataatgacaactgtatg
tgcaaatgtgccctcatgttaacaggaggatggtggtttgatgcttgtgg
cccctccaatctaaacggaatgttctatactgcggggcaaaaccatggaa
aactgaatgggataaagtggcactacttcaaagggcccagttactcctta
cgttccacaactatgatgattcgacctttagatttttga.

In another aspect, the COMP-Ang1 agent can include a homologue to the COMP-Ang1 protein that is either administered to, or endogenously expressed within, the eye of a subject. In one embodiment, the homologue can be at least 70% homologous to COMP-Ang1. In another embodiment, the homologue can be at least 80% homologous to COMP-Ang1. In another embodiment, the homologue can be at least 90% homologous to COMP-Ang1. In another embodiment, the homologue can be at least 95% homologous to COMP-Ang1.

In another embodiment, the COMP-Ang1 agent can include a precursor to the COMP-Ang1 protein that is either administered to, or endogenously expressed within, the eye of a subject. A COMP-Ang1 agent can also include a modified COMP-Ang1 protein or expression vector adapted to express a modified COMP-Ang1 protein. Any combination of the above mentioned COMP-Ang1 agents can also be used.

A therapeutically effective amount of a COMP-Ang1 agent can vary depending on the type of agent used. Where a COMP-Ang1 protein or homologue is used, a therapeutically effective amount can be from about 1 microgram to about 5 milligrams. In one aspect, the therapeutically effective amount can be from about 10 micrograms to about 2 milligrams. In one aspect, the therapeutically effective amount can be from about 50 micrograms to about 1 milligram.

Where an expression vector is used, the therapeutically effective amount can include from about 1×10⁷ to about 1×10¹² vector units. In another aspect, the therapeutically effective amount can include from about 2×10⁸ to about 1×10¹¹ vector units. In another aspect, the therapeucially effective amount can include from about 1×10⁹ to about 1×10¹¹ vector units.

The COMP-Ang1 agent and/or progenitor cells can be administered in a number of volumes of the composition. In one aspect, the therapeutically effective amount can include from about 0.001 ml to about 100 ml. In another aspect, the therapeutically effective amount can include from about 0.01 ml to about 5 ml. In another aspect, the therapeutically effective amount can include from about 0.01 ml to about 1 ml. In another aspect, the therapeutically effective amount can include from about 0.01 ml to about 0.1 ml. In another aspect, the therapeutically effective amount can include from about 0.001 ml to about 0.1 ml. In one specific aspect, intravitreal or other ocular administrations can include from 0.001 ml to 1 ml. In another specific aspect, systemic administration can include from about 5 to about 100 ml. In another aspect, the therapeutically effective amount can include from about 1 ml to about 20 ml. In one aspect, these ranges of volumes represent total volumes of the composition, whether the COMP-Ang1 agent is administered alone or jointly with progenitor cells. In another aspect, where progenitor cells are included in the composition, these ranges can be used for each of the COMP-Ang1 agent and the progenitor cells. In another aspect, progenitor cells can be administered separately from the COMP-Ang1 agent, but can be provided in the ranges of volumes listed above.

The current method can be used to treat a variety of subjects. In one aspect, the subject can include a mammal. In one aspect, the subject can be a human subject. In another aspect, the subject can be a veterinary subject, such as dogs, cats, cows, horses, deer, primates, rabbits, mice, hamsters, aquatic mammals, and the like.

Various treatment periods and regimens can be implemented with the current method. In one embodiment, the treatment period can occur during or after an ischemic or hypoxic event. In one embodiment the treatment period can occur prior to an ischemic or hypoxic event.

In one aspect, the therapeutically effective amount can be administered at least one time over a period of three weeks. In one aspect, the therapeutically effective amount can be administered up to 3 times over a period of three weeks. In one aspect, the therapeutically effective amount can be administered at least three times over a period of three weeks. In one aspect, the therapeutically effective amount can be administered at least one time every three weeks during the treatment period. In one aspect, the therapeutically effective amount can be administered up to three times every three weeks during the treatment period. In one aspect, the therapeutically effective amount can be administered at least three times every three weeks during the treatment period.

In one aspect, a treatment period can be six months, one year, 18 months, or 24 months. In one aspect, a single administration can be effective for about or at least six months, one year, 18 months, or 24 months.

In many cases, the exact onset of the ischemic or hypoxic event will be difficult to determine. However, the subject may recognize symptoms associated with an ischemic or hypoxic event, such as blurred vision, other visual impairment, or other ocular symptoms. Alternatively, a subject may not recognize the symptoms of the ischemic or hypoxic event, but a health care provider can recognize the symptoms of an ischemic or hypoxic event, diagnose the subject, and begin the treatment period within the time ranges previously listed. Accordingly, in one aspect, the treatment period can be initiated within about 72 hours of the onset of, recognition of, or diagnosis of an ischemic or hypoxic event. In another aspect, the treatment period can be initiated within about 48 hours of the onset of, recognition of, or diagnosis of an ischemic or hypoxic event. In another aspect, the treatment period can be initiated within about 24 hours of the onset of, recognition of, or diagnosis of an ischemic or hypoxic event. In another aspect, the treatment period can be initiated within about 12 hours of the onset of, recognition of, or diagnosis of an ischemic or hypoxic event. In another aspect, the treatment period can be initiated within about 8 hours of the onset of, recognition of, or diagnosis of an ischemic or hypoxic event.

Once the initial treatment is administered, the patient can continue to receive subsequent doses of the COMP-Ang1 agent as previously described. The treatment period for administering an initial dose can vary depending on the severity of the condition, such as complete verses partial occlusion. A more complete occlusion of the retinal artery can benefit from an initiation of the treatment period that occurs as early as possible.

In one aspect, the treatment period includes recurring treatment intervals, such as every week, every three weeks, every month, every six months, every year, every 18 months, every 24 months, and the like. In some aspects, a fixed number of treatment intervals can be included in each treatment period. In some aspects, the subject can be reassessed at the conclusion of each treatment interval to determine a need for extending the treatment period beyond a particular treatment interval. It is noted that the frequency of administration can depend on the patient, nature of the health condition, dosage and/or dosage form of the COMP-Ang1 agent, and the like. Amounts and frequency of administration can be optimized according to these and other relevant considerations known in the art.

A therapeutic construct for treating a disease or disorder of the eye is also described herein. The therapeutic construct can include a vector backbone contained in a viral particle. The vector backbone can include a sequence that is at least 70% homologous with SEQ ID 001. In another aspect, the vector backbone can include a sequence that is at least 80% homologous with SEQ ID 001. In another aspect, the vector backbone can include a sequence that is at least 90% homologous with SEQ ID 001. In another aspect, the vector backbone can include a sequence that is at least 95% homologous with SEQ ID 001.

In one aspect, the viral backbone can encode for a plurality of therapeutic agents or mediators. The plurality of therapeutic agents or mediators can be administered using bicistronic or multicistronic cassettes or vectors, such as an internal ribosome entry site (IRES) cassette, an A2 cassette, or other suitable cassette. Alternatively, the plurality of therapeutic agents or mediators can be administered via separate vectors contained in a common formulation. The plurality of therapeutic agents or mediators can include soluble VEGFR-1, nerve growth factor, ciliary neurotrophic factor, anti-PDGF-B proteins, anti-inflammatory proteins, the like, and combinations thereof.

These cassettes or vectors can be used with a variety of suitable viral particles, such as those previously listed. Further, whether monocistronic, bicistronic, or multicistronic, the therapeutic construct can be administered as part of a therapeutic composition or dosage form as a stand-alone therapeutic agent, or in connect with other therapeutic agents and/or stem or progenitor cells.

Accordingly, a composition or dosage form for treating a disease or disorder of the eye can include a therapeutically effective amount of a COMP-Ang1 agent, as previously described, and a pharmaceutically acceptable carrier. A number of suitable pharmaceutically acceptable carriers can be used. For example, a pharmaceutically acceptable carrier can include at least one of, or can include components selected from the group consisting of, a solubilizing agent, a tonicity agent, a pH adjuster, a stabilizing agent, a disaggregation agent, preservatives, the like, and combinations thereof.

Non-limiting examples of solubilizing agents can include phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus. Additionally, Ringer's lactate solution, normal saline (i.e. 0.9% saline), ½ normal saline, and the like. Combinations of the above mentioned solubilizing agents can also be used. Solubilizing agents can be present in the pharmaceutically acceptable carrier in various amounts. In one aspect, the solubilizing agent can have a concentration in the carrier of from about 10 wt %, about 20 wt %, about 30 wt %, or about 40 wt % to about 80 wt %, about 90 wt %, about 95 wt %, about 97 wt %, or 99 wt %.

Non-limiting examples of tonicity agents can include the solubilizing agents previously listed, as well as sodium chloride, potassium chloride, calcium chloride, magnesium chloride, mannitol, sorbitol, dextrose, glycerin, propylene glycol, ethanol, trehalose, and the like. The tonicity agent can be used to provide an appropriate tonicity of the formulation. In one aspect, the tonicity of the formulation is from about 250 to about 350 milliosmoles/liter (mOsm/L). In another aspect, the tonicity of the formulation is from about 277 to about 310 mOsm/L. Tonicity agents can be present in the pharmaceutically acceptable carrier in various amounts. In one aspect, the tonicity agent can have a concentration in the carrier of from about 1 wt %, about 5 wt %, about 10 wt %, or about 20 wt % to about 30 wt %, about 40 wt %, about 50 wt %, or about 60 wt %.

Non-limiting examples of pH adjusters can include a number of acids, bases, and combinations thereof, such as hydrochloric acid, phosphoric acid, citric acid, sodium hydroxide, potassium hydroxide, calcium hydroxide, and the like. The pH adjusters can be used to provide an appropriate pH for the formulation. In one aspect, the pH can be from about 5.5 to about 8.5. In one aspect, the pH can be from about 6 to about 8. In another aspect, the pH can be from about 6.5 to about 7.8. pH adjusters can be present in the pharmaceutically acceptable carrier in various amounts. In one aspect, the pH adjuster can have a concentration in the carrier of from about 0.01 wt %, about 0.05 wt %, about 0.1 wt %, or about 0.5 wt % to about 1 wt %, about 2 wt %, about 5 wt %, or about 10 wt %.

Non-limiting examples of stabilizing agents can include glycerol, propylene glycol, polyethylene glycol, copolymers of ethylene oxide and propylene glycol, such as Pluronic® F-68 NF Prill Poloxamer 188, Lutrol® L44, Lutrol® F87, and the like. Stabilizing agents can be present in the pharmaceutically acceptable carrier in various amounts. In one aspect, the stabilizing agent can have a concentration in the carrier of from about 0.01 wt %, about 0.05 wt %, about 0.1 wt %, or about 0.5 wt % to about 2 wt %, about 8 wt %, about 15 wt %, or about 30 wt %.

Non-limiting disaggregation agents can include the stabilizing agents previously listed as well as serum albumin, such as human serum albumin, ovalbumin, bovine serum albumin, and the like. Disaggregation agents can be present in the pharmaceutically acceptable carrier in various amounts. In one aspect, the disaggregation agent can have a concentration in the carrier of from about 0.01 wt %, about 0.05 wt %, about 0.1 wt %, or about 0.5 wt % to about 1 wt %, about 5 wt %, about 10 wt %, or about 20 wt %.

Non-limiting examples of preservatives can include benzalkonium chloride (BAK), cetrimonium, sodium perborate, ethylenediaminetetraaceticacid (EDTA) and its various salt forms, chlorobutanol, and the like. Preservatives can be present in the pharmaceutically acceptable carrier in various amounts. In one aspect, the preservative can have a concentration in the carrier of from about 0.001 wt %, about 0.005 wt %, about 0.01 wt %, or about 0.05 wt % to about 0.1 wt %, about 0.25 wt %, about 0.5 wt %, or about 1 wt %.

In one specific example, the pharmaceutically acceptable carrier includes serum albumin in an amount from about 1 wt % to about 10 wt %, PBS in an amount from about 5 wt % to about 75 wt %, BSS in an amount from about 15 wt % to about 75 wt %, and a copolymer of ethylene oxide and propylene oxide in an amount from about 0.25 wt % to about 4 wt %. In another specific example, the pharmaceutically acceptable carrier includes Ringer's lactate solution in an amount from about 5 wt % to about 90 wt % and normal saline in an amount from about 5 wt % to about 90 wt %.

The formulation or composition can also include penetration enhancers, thickeners, additional therapeutic agents, a therapeutically effective amount of progenitor or stem cells (as described previously), various other suitable additives known in the art, and combinations thereof.

In another embodiment, the composition can be included in a system and/or kit for treating a disease or disorder in the eye. The composition of the system can be any composition described herein. Specifically, the composition can include a therapeutically effective amount of a COMP-Ang1 agent and a pharmaceutically acceptable carrier. The system can also include a container.

As previously described, the composition of the system can also include a therapeutically effective amount of progenitor or stem cells. The composition can also be pre-mixed, such that it is ready to administer without further dilution. Alternatively, the composition can be prepared as a concentrate that can be diluted prior to administration.

The composition can be provided in a variety of containers depending on the desired format of administration. In one embodiment, the container can be a syringe. Where the composition includes progenitor or stem cells, the container can be a double-barreled syringe with the COMP-Ang1 agent and the progenitor cells each held separately, but then become mixed when administered to the subject's eye. In another embodiment, the container can be an amber colored container. In another embodiment, the container can include a material or can be made of a material selected from the group consisting of glass, polyethylene, polypropylene, and combinations thereof.

In one embodiment, the container can include a volume of the composition from about 0.01 ml to about 10 ml. In another embodiment, the container can include a volume of the composition from about 0.01 ml to about 5 ml. In another embodiment, the container can include a volume of the composition from about 5 ml to about 100 ml. In one aspect, the container can include a single dose of the composition. In another aspect, the container can include a plurality of doses of the composition.

As discussed further in the examples below, the COMP-Ang1 agent can normalize the retinal vasculature by decreasing Src phosphorylation, reducing VEGF-A expression, increasing VE-cadherin expression and stability, enhancing vascular integrity, and preventing endothelial cell loss. These processes can result in normalized vasculature, enhanced perfusion, reduced hypoxia-driven VEGF production (further contributing to vascular stability), and reduced ganglion cell layer loss. The decrease in VEGF production can derive from either COMP-Ang1 mediated decrease in retinal hypoxia or other related condition via inhibition of macrophage-produced VEGF.

Additionally, as illustrated in the examples below, COMP-Ang1 gene therapy can replace deficient Ang1 secretion by pericytes. A single intravitreous injection of AAV2.COMP-Ang1, affording long-term expression of COMP-Ang1, can preserve retinal morphology and prevent diabetes-induced deficits in visual acuity and retinal function in the retina. Furthermore, COMP-Ang1 can enhance ECFC integration into the retina of subjects with advanced diabetes, stemming further visual decline. This therapy suppresses the pathognomonic features of non-proliferative diabetic retinopathy and, in contrast to existing therapies, decreases the nonperfusion and ischemia critical to the genesis of proliferative diabetic retinopathy. COMP-Ang1 can prevent retinal vascular endothelial cell damage during diabetes. Moreover, enhanced expression of this vasotrophic growth factor enhances the established vasoregenerative properties of ECFCs delivered to the diabetic retina as cell therapy. COMP-Ang1 can be useful for vascular normalization in diabetic retinopathy and other conditions, which entail vascular ischemia (e.g., retinal vascular occlusions, stroke, heart disease, and peripheral vascular disease) or instability (e.g., macular degeneration, psoriasis, Crohn's disease, nephropathy, pulmonary edema, and rheumatoid arthritis).

Also as shown in the examples below, COMP-Ang1 can in some embodiments increase ECFC vasculogenic capabilities and promote their integration into and engraftment with the diabetic retinal vessels. This is functionally relevant because the combination of AAV2.COMP-Ang1 and ECFCs can stem further visual decline in mice with advanced diabetes.

The current technology can be illustrated through a few non-limiting examples, as follows:

In one example, a method of treating a disease or disorder of the eye is described. The example can include administering a therapeutically effective amount of a cartilage oligo matrix protein-Angiopoietin 1 (COMP-Ang1) agent to an eye of a subject during a treatment period.

In one example, the method can be a method of treating CRAO.

In one example, the method can be a method of preserving neural function in retinal tissue following a hypoxic or ischemic event.

In one example, the method can be a method of protecting neurons during a hypoxic event.

In one example, the method can be a method of preventing neurovascular dysfunction in an eye of a subject.

In one example, the method can be a method of reprogramming a Müller cell to function as a neural cell.

In one example, treating a disease or disorder of the eye includes reprogramming a Müller cell to function as a neural cell.

In one example, the disease or disorder of the eye is selected from the group consisting of neurovascular dysfunction, neuronal dysfunction, vascular hyperpermeability, retinal ischemia, retinal hypoxia, retinal hypoglycemia, retinal hyperglycemia, retinal stroke, central retinal artery occlusion (CRAO), central retinal vein occlusion, diabetic retinopathy, diabetic macular edema, pericyte dropout, endothelial cell dropout, capillary dropout, decreased blood-retinal barrier (BRB) integrity, leukocyte adhesion, inflammation, and the like.

In one example, the method further comprises administering a therapeutically effective amount of progenitor cells to an eye of the subject.

In one example, the therapeutically effective amount of progenitor cells is from about 5,000 cells to about 60,000,000 cells.

In one example, administering includes injection.

In one example, the COMP-Ang1 agent is a COMP-Ang1 protein or homologue thereof.

In one example, the therapeutically effective amount is from about 0.001 mg to about 5 mg of COMP-Ang1 protein or a homologue thereof.

In one example, the COMP-Ang1 agent is an expression vector that is configured to express a COMP-Ang1 protein or a homologue thereof.

In one example, the expression vector is a member selected from the group consisting of a lentivirus, an adenovirus, a cytomegalovirus, an adeno-associated virus (AAV), and combinations thereof.

In one example, the viral particle is an AAV which is a member selected from the group consisting of AAV2, AAV9, AAV10, and combinations thereof.

In one example, the viral particle is an AAV2.

In one example, the therapeutically effective amount is from about 1×10⁹ to about 1×10¹¹ vector units.

In one example, the therapeutically effective amount is from about 0.01 ml to about 1 ml.

In one example, the therapeutically effective amount is from about 5 ml to about 100 ml.

In one example, the subject is a human subject.

In one example, the subject is a veterinary subject.

In one example, the treatment period occurs during or after an ischemic or hypoxic event.

In one example, the treatment period is initiated within 72 hours of the onset of the ischemic or hypoxic event.

In one example, the treatment period occurs prior to an ischemic or hypoxic event.

In one example, the treatment period is a period of about 3 weeks.

In one example, a treatment regimen includes administering a therapeutically effective amount of the COMP-Ang1 agent up to three times during the treatment period.

In one example, the treatment period is about 6 months.

In one example, a single administration of the COMP-Ang1 agent is effective for at least 6 months.

In one example, a therapeutic construct for a disease or disorder of the eye is described. The therapeutic construct can include a vector backbone contained in a viral particle, the vector backbone including a sequence that is at least 80% homologous with SEQ ID 001.

In one example, the vector backbone encodes for a plurality of therapeutic agents or modifiers.

In one example, the viral particle is a member selected from the group consisting of a lentivirus, a cytomegalovirus, an adenovirus, an AAV, and combinations thereof.

In one example, the viral particle is AAV2.

In one example, a composition for treating a disease or disorder of the eye is described. The composition can include a therapeutically effective amount of a COMP-Ang1 agent and a pharmaceutically acceptable carrier.

In one example, the COMP-Ang1 agent is a COMP-Ang1 protein or homologue thereof.

In one example, the therapeutically effective amount is from about 0.001 mg to about 5 mg of COMP-Ang1 protein or a homologue thereof.

In one example, the COMP-Ang1 agent is an expression vector that is configured to express a COMP-Ang1 protein or a homologue thereof.

In one example, the expression vector is a member selected from the group consisting of a lentivirus, an adenovirus, a cytomegalovirus, an adeno-associated virus (AAV), and combinations thereof.

In one example, the viral particle is an AAV which is a member selected from the group consisting of AAV2, AAV9, AAV10, and combinations thereof.

In one example, the viral particle is an AAV2.

In one example, the therapeutically effective amount is from about 1×10⁹ to about 1×10¹¹ vector units.

In one example, the therapeutically effective amount is from about 0.01 ml to about 10 ml.

In one example, the therapeutically effective amount is from about 5 ml to about 100 ml.

In one example, the pharmaceutically acceptable carrier includes at least one of a solubilizing agent, a tonicity agent, a pH adjuster, a stabilizing agent, a disaggregation agent, and combinations thereof.

In one example, the pharmaceutically acceptable carrier includes at least one of Ringer's lactate, normal saline, phosphate-buffered saline (PBS), a balanced salt solution, albumin, and a copolymer of ethylene oxide and propylene oxide.

In one example, the pharmaceutically acceptable carrier comprises Ringer's lactate and normal saline.

In one example, the pharmaceutically acceptable carrier comprises albumin, PBS, balanced salt solution, and a copolymer of ethylene oxide and propylene oxide.

In one example, the pH is from about 6.5 to about 7.8.

In one example, the tonicity is from about 277 to about 310 mOsm/L.

In one example, the composition further comprises a therapeutically effective amount of progenitor cells.

In one example, the therapeutically effective amount of progenitor cells is from about 5,000 cells to about 60,000,000 cells.

In one example, a system for treating disease or disorder in an eye of a subject is described. The system can include a composition and a container. The composition can include a therapeutically effective amount of a COMP-Ang1 agent and a pharmaceutically acceptable carrier.

In one example, the composition further comprises a therapeutically effective amount of progenitor cells.

In one example, the composition is a pre-mixed composition that is ready to administer without further dilution.

In one example, the container is an amber-colored container.

In one example, the container is made a material selected from the group consisting of glass, polyethylene, polypropylene, and combinations thereof.

### Examples

The following examples illustrate some embodiments of the current technology. However, it is to be understood that the following are only exemplary or illustrative of the application of the principles of the present compositions, systems, and methods. Numerous modifications and alternative compositions, systems, and methods may be devised without departing from the spirit and scope of the present disclosure. The appended claims are intended to cover such modifications and arrangements. Thus, while the examples have been described above with particularity, the following provide further detail in connection with only a few particular examples.

Mice are used in several of the following examples. Specifically, the diabetic C57BL/6-Ins2^{Akita}/J (Ins2Akita) and its background wild type (WT) strain, C57BL6/J, were used. Mice heterozygous for the Ins2 mutation can experience hypoinsulinemia and hyperglycemia by four weeks of age and progressive retinal abnormalities 12 weeks after the onset of hyperglycemia, which include apoptosis (i.e. endothelial and RGC loss) and functional deficits (increased vascular permeability and decreased neuronal function). Thus, as a diabetic mouse model, the Ins2Akita mouse exhibits several hallmarks of non-proliferative diabetic retinopathy including vascular hyperpermeability and inflammation.

Genotyping was performed following The Jackson Laboratory protocol. Blood sugar level was measured by using OneTouch® Ultra®. Only male Ins2Akita with blood sugar levels greater than 600 mg/dL or age-matched controls were used.

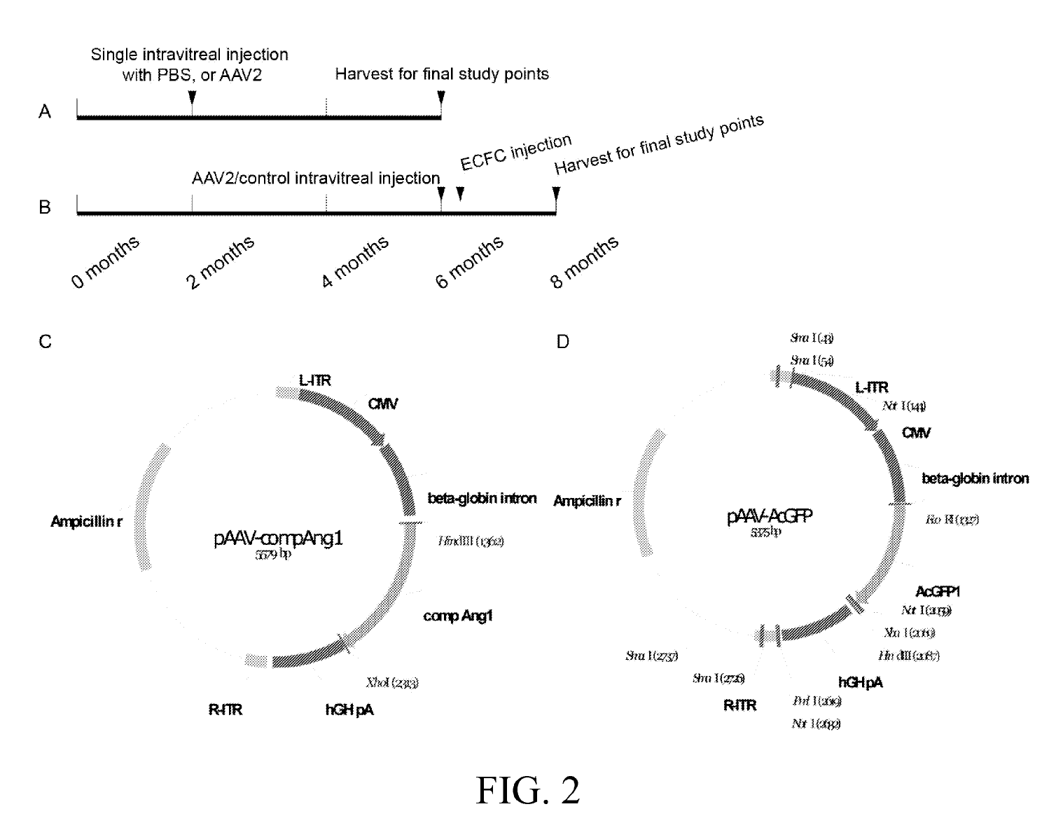

In various examples, Mice were randomly assigned to one of three experimental groups: AAV2.COMP-Ang1, AAV2.AcGFP (Aequorea coerulescens green fluorescent protein), or phosphate-buffered saline (PBS). At 2 months of age (FIG. 2A), each mouse was anesthetized by intraperitoneal injection of 1.25% tribromoethanol at a dose of 0.025 mL/gram of body weight. Each mouse was treated with either 2 μL of AAV2 solution (2.0×10⁹ particles) or PBS injected into the vitreous cavity of both eyes with a 33-gauge microsyringe. An additional cohort of mice was treated as described above at 6 months of age. Two weeks later, this cohort received a second intravitreal injection with 1 μL of 1×10⁵ECFCs (FIG. 2B).

Further, several of the following examples utilize an expression vector to deliver the COMP-Ang1 agent to the eye of the subjects. To construct the vectors, the plasmids pAAV.COMP-Ang1 and pAAV.AcGFP were created by incorporating the COMP-Ang1 cDNA from the pCMV-dhfr2-COMP-Ang1 into pAAV-MCS (FIG. 2C). pAAV.AcGFP was created by the same technique with AcGFP cDNA from the pIRES2-AcGFP1 plasmid (FIG. 2D).

For in vivo assays requiring imaging with the Spectralis HRA+OCT (Heidelberg Engineering), mice were anesthetized by an inhalation of 3% isoflurane/O₂ mixture in a closed canister at a flow rate of 1.0 Lpm. Pupils were dilated with a 1% tropicamide.

Retinas were harvested and processed for in situ hybridization (ISH) according to standard protocols using procedures to avoid RNAse contamination.

During Reverse Transcriptase Polymerase Chain Reaction (RT-PCR) for COMP-Ang1 mRNA Expression, the following primer sequences for COMP-Ang1 were used:

COMP-Ang1 F 5′-GCTCTGTTTTCCTGCTGTCC-3′
COMP-Ang1 R 5′-GTGATGGAATGTGACGCTTG-3′

Primer sequences for the internal control, glyceraldehyde 3-phosphate dehydrogenase (GAPDH) were:

GAPDH F 5′-AACTTTGGGATTGTGGAAGGG-3
GAPDH R 5′-ACCAGTGGATGCAGGGATGAT-3′

All numerical data were analyzed in Excel (Microsoft, Redmond, Wash.) and presented as the mean±standard deviation (stdev). Student's two-tailed t-test, with a level=0.05, was used to compare differences between 2 samples. ANOVA test, with p<0.05, followed by Tukey post hoc analysis, was used to compare differences between 4 groups.

### Example 1: Intravitreal AAV2 Gene Therapy is Safe for the Mouse Retina and can Preserve Retinal Neurophysiological Function

Mice were dark-adapted, anesthetized with ketamine/xylazine (90 mg/10 mg per kg body weight), and placed on a controlled warming plate (TC-1000, CWE Instruments, Ardmore, Pa.). Electroretinograms (ERGs) were taken between a gold corneal electrode and a stainless-steel scalp electrode with a 0.3- to 500-Hz band-pass filter (UTAS E-3000, LKC Technologies, Gaithersburg, Md.). The photoflash unit was calibrated to deliver 2.5 cd s/m² at 0 dB flash intensity and scotopic measurements were recorded with flash intensities increasing from 0.0025 to 250 cd s/m². The b-wave amplitudes were determined in scotopic conditions, and the mean values at each stimulus intensity were compared with an unpaired two-tailed t-test.

Additionally, optomotor reflex-based spatial frequency threshold tests were conducted in a visuomotor behavior measuring system (OptoMotry, CerebralMechanics, Lethbridge, AB, Canada). Tracking was defined as a reproducible smooth pursuit with a velocity and direction concordant with the stimulus. Trials of each direction and spatial frequency were repeated until the presence or absence of the tracking response were established unequivocally. Rotation speed (12°/s) and contrast (100%) were kept constant.

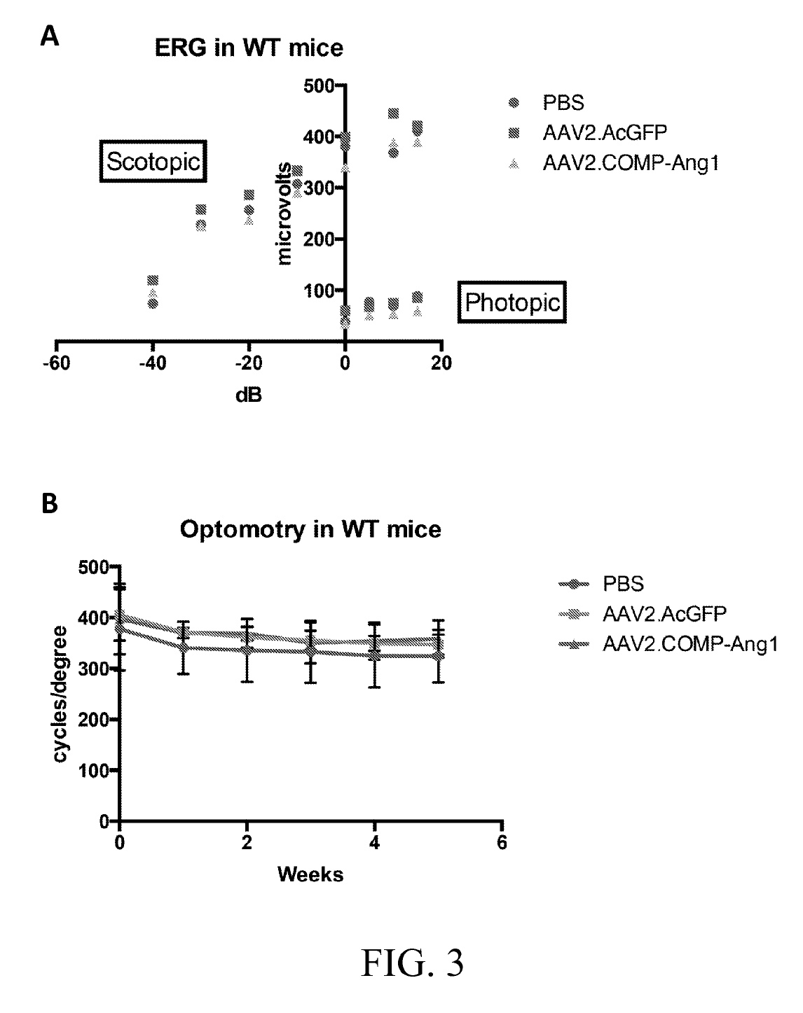

The present study found no significant differences (p=0.3 at −30 dB, 0.6 at 0 db, and 0.6 at 20 db) in scotopic and photopic b-wave amplitudes on ERG between WT mice treated with intravitreal AAV2.COMP-Ang1, AAV2.AcGFP, or PBS (FIG. 3A) Furthermore, optokinetic tracking (OKT) response of no injection WT control mice did not change appreciably compared to PBS-treated mice (p=0.5) over the course of 5 weeks (FIG. 3B).

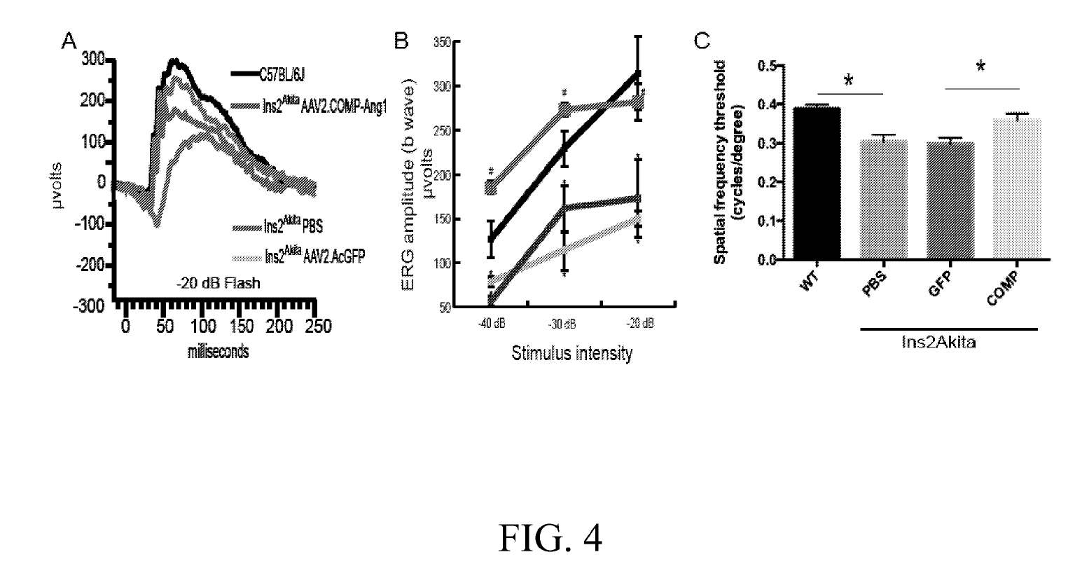

Additionally, patients with DR manifest with visual deficits early in the disease, and animals exhibit changes in visual acuity and contrast sensitivity through impaired visual tracking behavior and delayed retinal electrical responses. In line with this, ERG and OKT responses were within normal limits for WT mice but abnormally depressed in diabetic control mice (at −40 dB, WT=127, PBS=57, AAV2.AcGFP=73 volts, p<0.01; at 4 months post-injection, WT=0.388, PBS=0.184, AAV2.AcGFP=0.174 cycles/degree, p<0.01; FIG. 4A-C).

AAV2.COMP-Ang1 treatment diminished the dampening in scotopic b-wave amplitudes (185 volts, p<0.01, FIGS. 4A and B) caused by anomalous photoreceptor-bipolar communication in DR. Likewise, Ins2Akita mice treated with AAV2.COMP-Ang1 were able to avert the deterioration of OKT (0.312 cycles/degree; p<0.01; FIG. 4C).

Together, these data show that AAV2.COMP-Ang1 is safe and can preserve retinal neurophysiological function.

### Example 2: Intravitreal AAV2.COMP-Ang1 Expresses COMP-Ang1 in the Mouse Retina

Retinas were harvested and protein lysate samples were immunoprecipitated with anti-FLAG M2 affinity gel (Sigma-Aldrich). Eluted proteins samples were run on 12% SDS-PAGE. Overall protein levels were compared with anti-GAPDH (1:3000, Abcam, Cambridge, Mass.).

Enucleated globes were fixed in 4% paraformaldehyde (PFA) followed by retinal dissection. Specimens were stained with 1:200 alpha-smooth muscle actin (α-SMA) antibody or neuron-glial antigen 2 (NG-2) antibody conjugated with cyanine dye (Cy3) (Sigma-Aldrich) and 5 μg/ml AlexaFluor 647 conjugated isolectin GS-IB4 (Invitrogen) in blocking buffer overnight at 4° C. After washing, the retina was flat-mounted on a glass slide. Full retinal field immunofluorescence (IF) images were captured at low magnification, followed by increasing magnification of each quadrant with scanning laser confocal microscopy (Olympus America).

AAV2 localization and transfection in the retina was confirmed by in vivo and ex vivo detection of the fluorescent gene product of the sham viral control by confocal microscopy; further, COMP-Ang1 mRNA and protein expression was verified by ex vivo immunoassay.

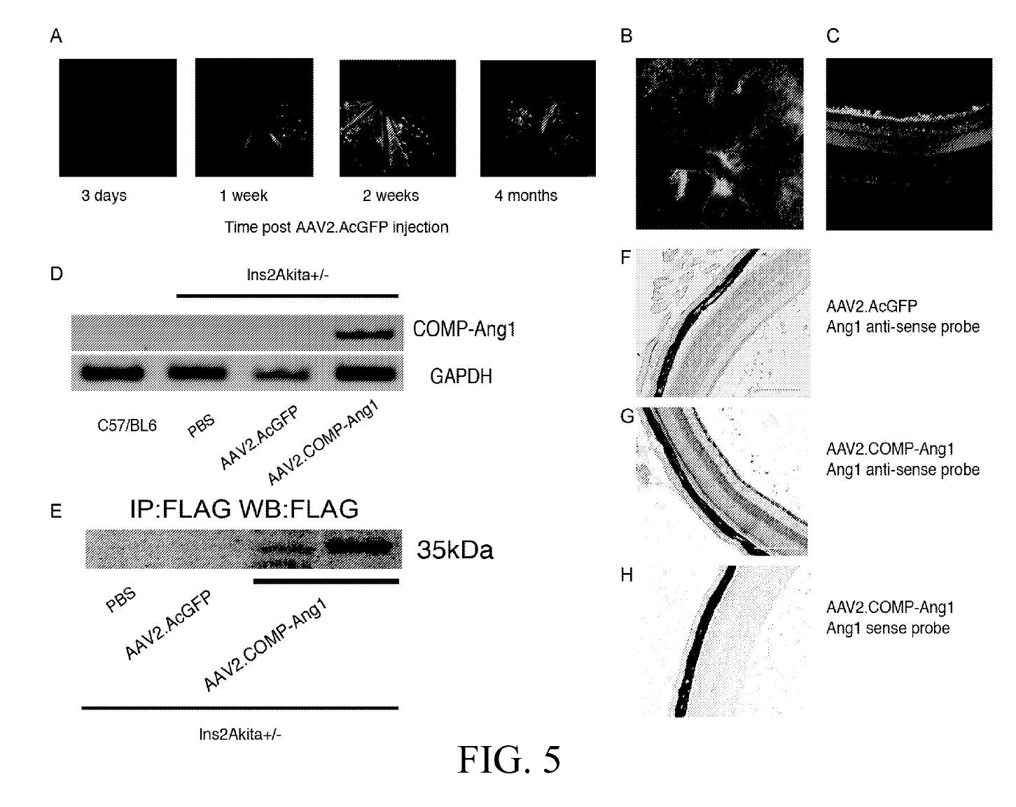

AcGFP fluorescence was initially observed at 1 week post-injection and persisted through to the 4 months post-injection endpoint (FIG. 5A). As shown in FIGS. 5B-C, AcGFP signal was visualized in all retinal quadrants at the level of the ganglion cell-inner plexiform layer (GC-IPL).

COMP-Ang1 production in the mouse retina was demonstrated via semi-quantitative reverse transcriptase polymerase chain reaction (RT-PCR) (FIG. 5D), in situ hybridization (ISH) (FIGS. 5F-H), and immunoblotting (FIG. 5E) at the 4 months post-injection endpoint. In parallel with AcGFP fluorescence, ISH revealed increased amounts of COMP-Ang1 mRNA in the inner retina, predominantly in the GC-IPL (FIGS. 5F-H).

### Example 3: COMP-Ang1 Prevents Breakdown of Vascular Structure

Retinas were harvested and protein lysate samples were immunoprecipitated with anti-FLAG M2 affinity gel (Sigma-Aldrich). Eluted proteins samples were run on 12% SDS-PAGE. Overall protein levels were compared with anti-GAPDH (1:3000, Abcam, Cambridge, Mass.) and anti-β-actin (1:3000, Abcam). Samples were tested for anti-VEGF-A (1:200, Santa Cruz Biotechnology, Santa Cruz, Calif.), anti-VE cadherin (1:1000, Abcam), and anti-phospho-Src (PY419, 1 μg/mL, R&D Systems).

For immunofluorescence (IF) of the retinal flat-mounts, enucleated globes were fixed in 4% paraformaldehyde (PFA) followed by retinal dissection. Specimens were stained with 1:200 alpha-smooth muscle actin (α-SMA) antibody or neuron-glial antigen 2 (NG-2) antibody conjugated with cyanine dye (Cy3) (Sigma-Aldrich) and 5 μg/ml AlexaFluor 647 conjugated isolectin GS-IB4 (Invitrogen) in blocking buffer overnight at 4° C. After washing, the retina was flat-mounted on a glass slide. Full retinal field immunofluorescence (IF) images were captured at low magnification, followed by increasing magnification of each quadrant with scanning laser confocal microscopy (Olympus America).

For trypsin digest to determine retinal vascular architecture, enucleated globes were fixed in 2% formalin. The retina was detached around the sub-retinal space. The optic nerve was cut under the disc. The specimen was digested at 37° C. in 2.5% trypsin/0.2M TRIS at pH 8.0 for 30-60 minutes. Specimens were transferred to distilled water and to 0.5% triton X-100 surfactant/distilled water and left at room temperature for another hour. Lastly, the specimen was moved to 0.1% triton X-100/distilled water for mounting and dried in a 37° C. incubator. Samples were stained with periodic acid Schiff staining and imaged with light microscopy (Olympus, Center Valley, Pa.). Acellular capillaries and pericytes were counted in each of a total of 5 high-powered fields (HPFs) per retinal quadrant.

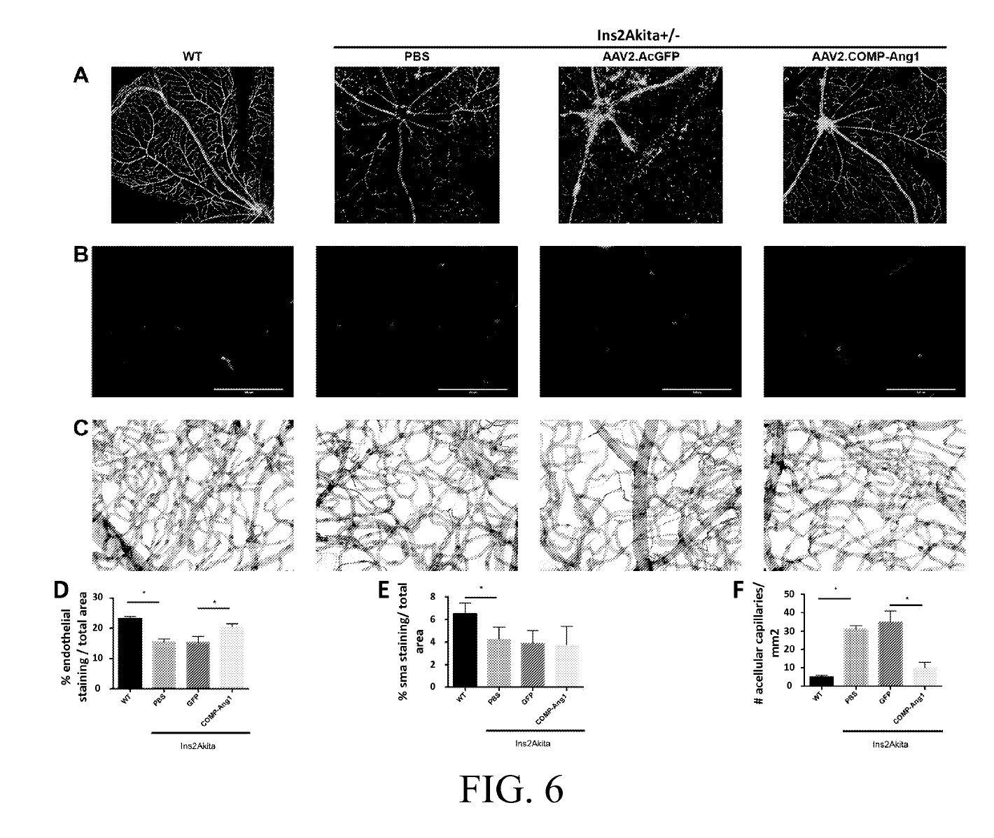

The principal morphologic features of DR are pericyte, endothelial cell, and capillary dropout. Concordantly, retinal vessel density was discernably reduced in immunofluorescence (IF) and trypsin digest images of Ins2Akita control (PBS and AAV2.AcGFP) versus WT retinas (FIGS. 6A-C). Endothelial cell (p<0.01, FIG. 6D) and pericyte (p<0.01, FIG. 6E) coverage were significantly decreased in the diabetic controls compared to WT, while capillary acellularity was increased (p<0.01, FIG. 6F).

IF and trypsin digest images of AAV2.COMP-Ang1 exhibit an improvement in vessel density compared to diabetic controls (FIG. 6A-C), accompanied by a significant drop in acellular capillary density (p<0.01 vs. AAV2.AcGFP, p<0.01 vs. PBS, and p<0.01 vs. WT, FIG. 6F). AAV2.COMP-Ang1 significantly rescued endothelial cell loss relative to controls (WT=23.2%, AAV2.COMP-Ang1=20.5%, PBS=15.5%, AAV2.AcGFP=15.3%, p<0.01 FIG. 6D), but not pericyte coverage (WT=6.5%, AAV2.COMP-Ang1=3.8%, PBS=4.3%, AAV2.AcGFP=3.9%, p=0.9, FIG. 6E). These data suggest that, in lieu of pericytes, AAV2.COMP-Ang1 can provide sufficient Ang1 endothelial trophic signaling to prevent capillary dropout (FIG. 6F).

### Example 4: COMP-Ang1 Promotes BRB Integrity

In vitro measurements of transendothelial electrical resistance (TER) were performed with an electrical cell-substrate impedance sensing (ECIS) system (Applied Biophysics, Troy, N.Y.). Human retinal microvascular endothelial cells (HrMVECs) (Cell Systems, Kirkland, Wash.) were seeded (50,000 cells/well) onto fibronectin-coated gold microelectrodes in ECIS culture wells (8W10E+, Applied Biophysics) and incubated overnight at 37° C. in complete medium (EBM-2+EGM2-MV, Lonza Group, Basel, Switzerland) until cell resistance reached a plateau. Cells were serum-starved for one hour until resistance was stabilized (1200Ω). Each well received one of three experimental treatments: COMP-Ang1 protein (Enzo Life Sciences, Farmingdale, N.Y.), VEGF protein (R&D, Minneapolis, Minn.), or PBS. Monitoring was continued for 21 hours. The data from triplicate wells were averaged and presented as normalized resistance versus time.

Mice were administered Evans blue (EB) (Sigma-Aldrich) at a dosage of 20 mg/kg through tail vein injection. After 4 hours, the vasculature was perfused with PBS. The retinas were next harvested and placed in formamide at 70° C. for 18 hours. Samples were centrifuged for 2 hours at 40,000 g in a 0.2 μm filter. EB concentration was detected spectrophotometrically by subtracting absorbance at 620 nm from 740 nm.

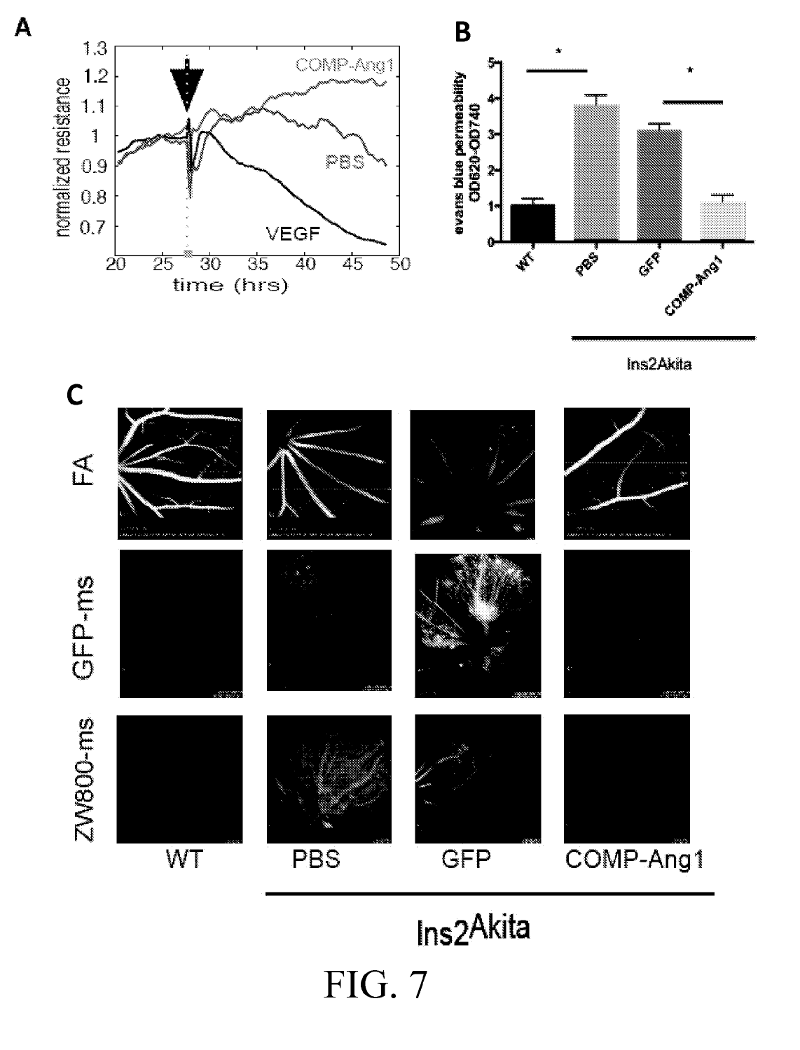

Ischemia from capillary dropout in DR stimulates VEGF production and consequent vascular hyperpermeability. Accordingly, the transendothelial electrical resistance (TER) of HrMVECs decreased after treatment with VEGF compared to PBS in vitro (p<0.01, FIG. 7A), while EB extravasation was elevated in PBS and AAV2.AcGFP treated diabetic controls (3.8 and 3.1 fold respectively, FIG. 7B) compared to WT mice (p<0.01). Microsphere leakage was similarly elevated in diabetic controls (FIG. 7C)

COMP-Ang1 significantly increased the TER (p<0.01, FIG. 7A) of HrMVECs and decreased EB extravasation (p<0.01, FIG. 7B) and microsphere (FIG. 7C) leakage in diabetic mice.

These results indicate that COMP-Ang1 restores the barrier function of the retinal vasculature in DR. To explore the molecular underpinnings of this finding, the influence of COMP-Ang1 on VEGF-A, the proto-oncogene non-receptor tyrosine kinase Src, and the intercellular junction adhesion molecule VE-cadherin were investigated. VEGF-A can induce vessel leakage in DR through Src-mediated downregulation of VE-cadherin, while Ang1 can upregulate VE-cadherin.

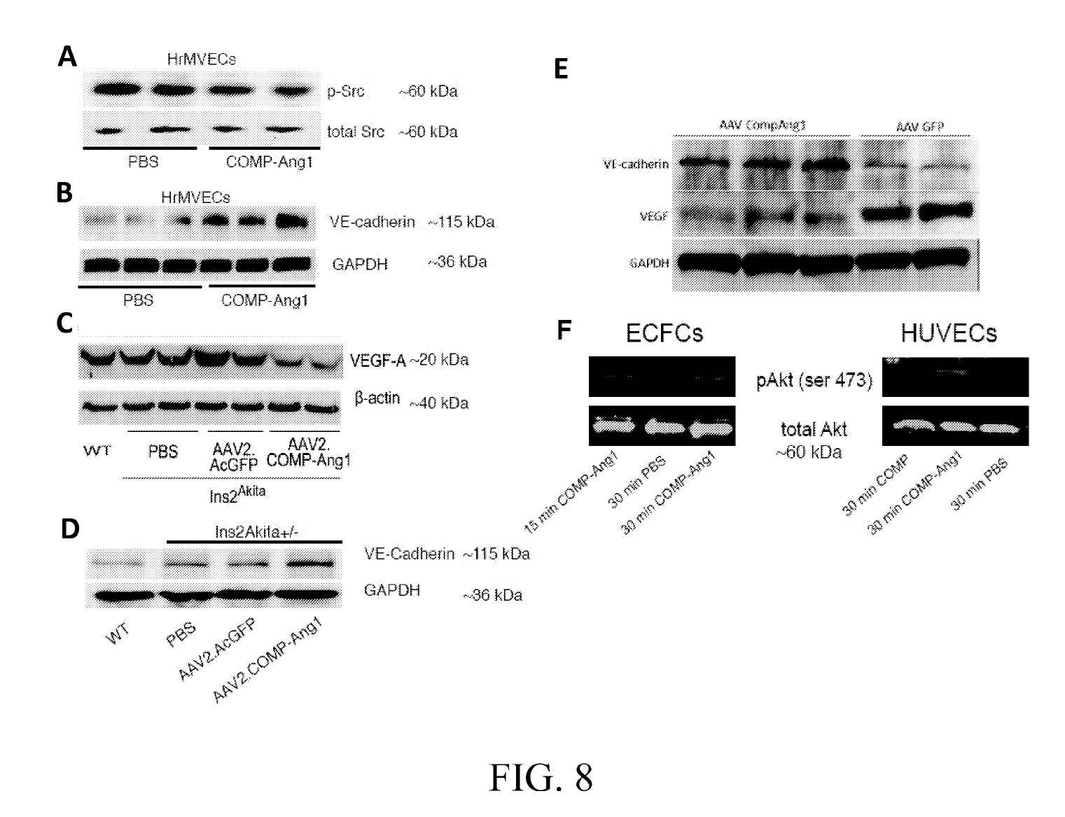

COMP-Ang1 decreased Src phosphorylation (FIG. 8A) and increased VE-cadherin expression in HrMVECs (FIG. 8B). Both Ins2Akita and WT retinas treated with AAV2.COMP-Ang1 had reduced levels of VEGF-A (FIGS. 8C, 8E) and increased levels of VE-cadherin (FIGS. 8D, 8E).

Ang1 acts on the PI3K/Akt cascade to prevent the apoptosis of damaged vascular endothelial cells, considering the reduction in capillary acellulatiry and endothelial cell loss with COMP-Ang1, Akt phosphorylation was explored as a possible mechanism for COMP-Ang1-mediated survival of endothelial cells. COMP-Ang1 increased Akt phosphorylation at the serine 473 residue in both ECFCs and human umbilical vein endothelial cells (HUVECs) (FIG. 8F).

### Example 5: COMP-Ang1 Reduces Leukocyte Endothelial Adhesion and Leukostasis

Acridine orange (AO (Acros Organics, Geel, Belgium), 0.10%/PBS) was filtered with a 0.22 μm filter. The solution (0.05 mL/min for a total of 1 minute) was injected into the tail vein. Imaging utilized Spectralis HRA+OCT (Heidelberg Engineering, Heidelberg, Germany) with a 488 nm argon blue laser with a standard 500 nm long-pass filter. Images were acquired from both eyes with a 55-degree lens utilizing the movie mode on the Spectralis HRA+OCT (Heidelberg Engineering).

HrMVECs were cultured in parallel-plate fibronectin-coated flow chambers (μ-Slide VI 0.4, ibidi USA, Madison, Wis.) until 80% confluent and exposed to tumor necrosis factor alpha (TNF-α) (long/mL) (R&D Systems, Minneapolis, Minn.) or vehicle control for 3 hours. Human leukocytes were isolated as described previously in accordance with Institutional Review Board guidelines and diluted in warmed ultrasaline (Lonza Group) to 1×10⁶ cells/ml. Leukocytes were pumped through the parallel plate flow chambers using a syringe pump (Harvard Apparatus, Holliston, Mass.) at 1 dynes/cm² (typical venous shear stress). Differential interference contrast (DIC) images were acquired at a rate of 1/second for 1 minute, and the number of leukocytes adhered to or rolling on the monolayer was quantified as leukocytes/frames/second. Three independent flow wells were averaged to attain the reported values.

DR is characterized by a chronic, subclinical inflammatory response that is thought to play a critical role in its pathogenesis. The less deformable and more activated leukocytes in DR are conjectured to contribute to retinal nonperfusion and capillary dropout through increased attachment to endothelial cells and entrapment within the capillaries.

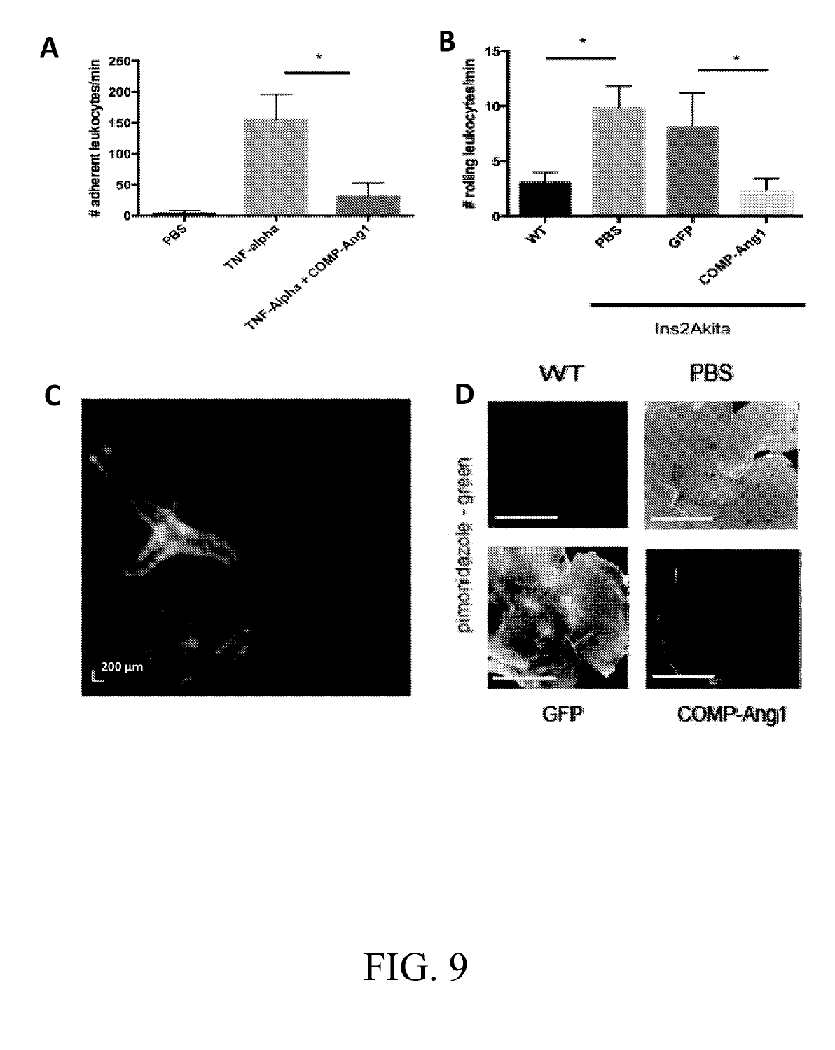

Leukocyte adhesion to the vascular wall is mediated, in part, by TNF-α. Correspondingly, the endothelial monolayer of HrMVECs exposed to TNF-α experienced an abnormally high rate of leukocyte adherence. Treatment with COMP-Ang1 protein decreased the number of adherent leukocytes per minute by 80% (p<0.01, FIG. 9A).

On AO leukocyte fluorography, leukocyte rolling was significantly elevated in Ins2Akita control (9.8 cells/min) versus WT retinas (3 cells/min, p<0.01, quantitative image FIG. 9B, representative image FIG. 9C showing leukocyte aggregations at the bifurcation). AAV2.COMP-Ang1 was able to reduce this rate to below the disease-free baseline (2.8 cells/min, p<0.01, FIG. 9B).

These results indicate that the improvement in vascular parameters by COMP-Ang1 may have an anti-inflammatory component.

### Example 6: COMP-Ang1 Reduces Hypoxia

Mice received an intraperitoneal injection of the bio-reductive hypoxia marker pimonidazole (Hypoxyprobe, Burlington, Mass.) at 60 mg/kg. Three hours later, retinas were harvested and stained with a hypoxyprobe-1 monoclonal antibody conjugated to fluorescein isothiocyanate (FITC) to detect reduced pimonidazole adducts (Hypoxyprobe) forming in pO₂<10 mmHg.

Leukostasis has been proposed as a mechanism of capillary non-perfusion and retinal hypoxia. Since hypoxia is a potent inducer of VEGF-A, the relationship between COMP-Ang1 and hypoxia was assessed. Pimonidazole staining was increased in the diabetic control mice relative to WT mice whereas it was reduced nearly to baseline levels in AAV2.COMP-Ang1 mice (FIG. 9D).

Collectively, these outcomes demonstrate a pathway for COMP-Ang1-mediated retinal vascular functional stabilization. The inhibition of leukocyte adhesion and stimulation of Akt phosphorylation lead to the preservation of perfusion and normalization of tissue oxygenation, whereas the inhibition of VEGF-A and Src phosphorylation lead to the preservation of VE-cadherin and normalization of permeability.

### Example 7: COMP-Ang1 Prevents Retinal Neuronal Dysfunction

Mice were imaged bilaterally with optical coherence tomography (OCT) (Spectralis HRA+OCT, Heidelberg Engineering). Retinal cross-sectional thickness was measured 250 μm relative to the optic nerve head, using the en face image as a guide. Measurements were recorded for each retinal quadrant and averaged to attain the reported values.

Each mouse received a dosage of 100 μL/20 gram tail vein injections with 100 nm microspheres (Magsphere, Pasadena, Calif.) conjugated to either the near infrared fluorophore ZW800 (Flare Foundation, Boston, Mass.) (4) or GFP (Magsphere). Bilateral imaging by Spectralis HRA+OCT (Heidelberg Engineering) was performed with FA and indocyanine green (ICG) modalities.

Enucleated globes were fixed in 4% PFA. The globes were cut in 10 μm sections and stained with anti-VE cadherin antibody (1:200, Abcam) and 4′,6-diamidino-2-phenylindole (DAPI) (Sigma-Aldrich). Sections were captured with scanning laser confocal microscopy (Olympus America).

4-month-old mice received intravitreal injections of either AAV2.COMP-Ang1 or PBS as described above. 6 months later, retinas were fixed and dissected as described above for flat mounts. Specimens were labeled with 1:200 pan-Brn3 antibody (Santa Cruz Biotechnology), followed by AlexaFluor 546 conjugated secondary antibody (Invitrogen), and counterstained with DAPI (Sigma-Aldrich). Eight fields were imaged for each retina using the 40× oil objective; these comprised four evenly spaced fields (one per quadrant) adjacent to the optic nerve and four fields (similarly spaced) near the flat mount periphery. Images were counted blind by two separate investigators. Counts were averaged for each retina and compared across control and experimental groups.

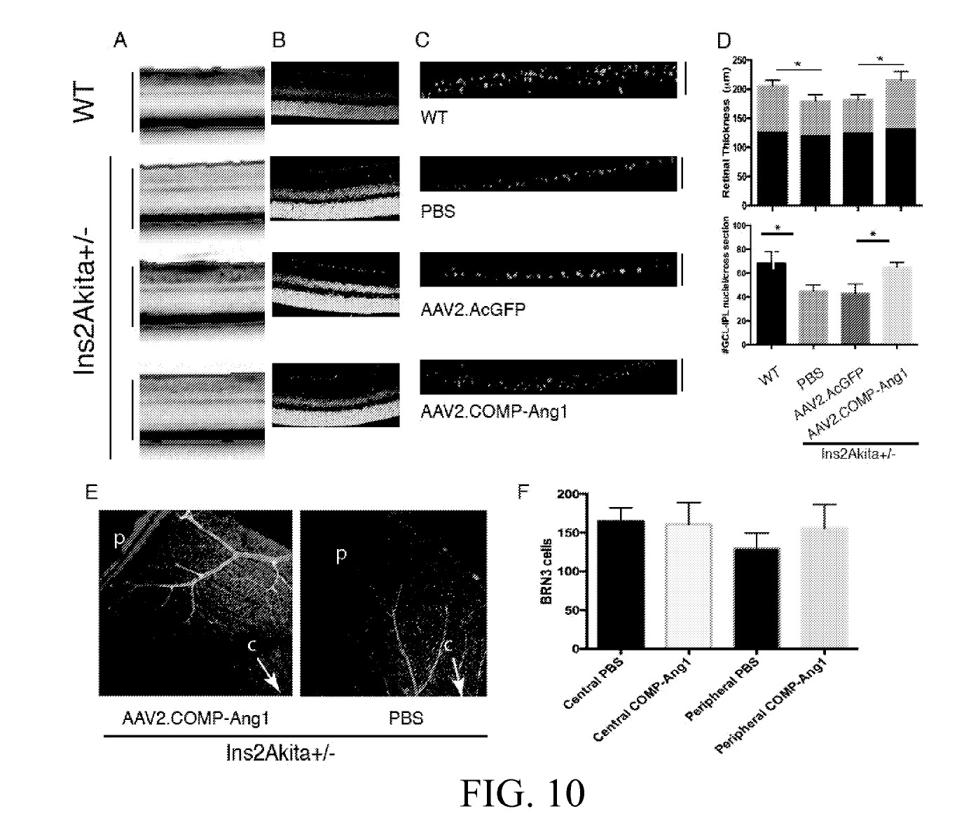

DR causes neural degeneration of the inner retina. Consistent with this, the retinas on optical coherence tomography (OCT) and IF images from Ins2Akita control mice (185 μm) were qualitatively and quantitatively thinner than WT mice (210 μm, p<0.01, FIGS. 10A-D). In parallel, GC-IPL cell density was also decreased in Ins2Akita control (68 nuclei/300 μm length) versus WT (45 nuclei/300 μm length) retinas (p<0.001, FIG. 9B-C), a 34% loss of cells.

AAV2.COMP-Ang1 preserved retinal thickness (WT=210 μm; AAV2.COMP-Ang1=205 AAV2.AcGFP=181 μm, PBS=185 μm; p<0.01; FIGS. 10A-D) and prevented loss of cells in the GC-IPL (65 nuclei/300 μm length, p=0.03) in Ins2Akita mice (FIG. 10D).

Further characterization of GC-IPL cells with the RGC-specific marker, Brn3, revealed no difference in RGC counts within the central retinas of PBS versus COMP-Ang1 treated Ins2Akita mice (p=0.7, (FIG. 10E). However, peripheral retinas showed a 17% loss of ganglion cells (FIG. 10F). Although this difference was not statistically significant (p=0.07), the trend shows an effect size similar to that reported for RGC loss in the peripheral retina of diabetic versus WT mice, suggesting that a larger sample size can provide sufficient power to confirm an effect.

In sum, these data suggest that AAV2.COMP-Ang1 is beneficial in preventing diabetes-induced GC-IPL atrophy and peripheral RGC cell loss, but may also target or recruit non-RGC cell types within the inner retina for neuroprotection.

### Example 8: COMP Ang1 Enhances ECFC Treatment Effect

The mice used, methods of sample preparation, and analytical methods were performed in accordance descriptions provided in the previous examples. Population doubling was carefully monitored, and ECFCs at early passages were used for all experiments. Three different clones of ECFCs were used for each experiment and results presented are representative images from one of the clones, while quantification data presented are representative of all clones tested. For in vivo testing, one clone type was chosen and used in all mice.

Fresh human cord blood underwent density gradient fractionation for the isolation of mononuclear cells (MNCs) and were selected for ECFCs via resuspension in complete medium (EBM+EGM-2 MV, Lonza Group) supplemented with 10% FBS and seeding onto 24-well culture plates pre-coated with rat tail collagen type 1 (BD Biosciences, Bedford, UK) at a density of 1×10⁷ cells/mL. Cells were labeled (Qtracker 655, Invitrogen, Life Technologies, Carlsbad, Calif.) per manufacturer's instructions.

From the mice which had received intravitreal ECFCs, harvested retinas were fixed and dissected as described above for flat mounts (Supplemental FIG. 3). Specimens were stained with 5 μg/ml AlexaFluor 647 conjugated isolectin GS-IB4 (Invitrogen) and mounted on a glass slide as described above. ECFC integration was counted in 4 high-powered fields in each retinal quadrant.

ECFCs were plated on rat-tail collagen-coated 6 well plates pre-labeled with traced lines (27). When cells were 90% confluent, a uniform straight scratch was made in the monolayer using a 200 μl pipette tip. After injury, cells were washed, medium was changed, and reference photographs were taken within each region marked by the lines using a phase contrast microscope (Eclipse E400, Nikon, Tokyo, Japan). Wells were incubated with the experimental treatment (doses of COMP-Ang1 protein from 0-1000 ng/mL) and images were captured at hourly intervals. Endothelial cell migration was quantified by calculating the proportion of denuded area.

ECFCs were labeled with a fluorescent dye (PKH Cell Linker Kit for General Cell Membrane Labeling, Sigma-Aldrich) as previously described. Next, they were suspended in growth factor reduced basement membrane matrix (Matrigel, Becton Dickinson Biosciences, Franklin Lakes, N.J.) and 50 μL aliquots were spotted onto a 24-well plate. Spots were covered with DMEM containing 5% porcine serum and treated with either control or increasing doses of COMP-Ang1. After 24 hours, wells were assessed for the presence of tubules. Images were acquired by using a laser confocal microscope (Nikon).

Many diabetics present with advanced retinal vasculature loss. The recellularization and resultant refunctionalization of acellular capillaries can halt the nonperfusion at the root of DR pathophysiology, but in diabetes the endogenous reparative cells responsible for this task have a decreased ability to associate with existing vascular networks. The exogenous delivery of human-derived ECFCs, as evidenced by their utility in oxygen-induced retinopathy, may be able to compensate for this deficiency but have not yet been explored in the context of DR. ECFCs can express high levels of the Ang1 receptor, Tie2, and Ang1 can promote the differentiation of stem cells into vasculogenic cells for vessel engraftment and reformation. Therefore, the regenerative potential of dual therapy with COMP-Ang1 and ECFCs was also tested.

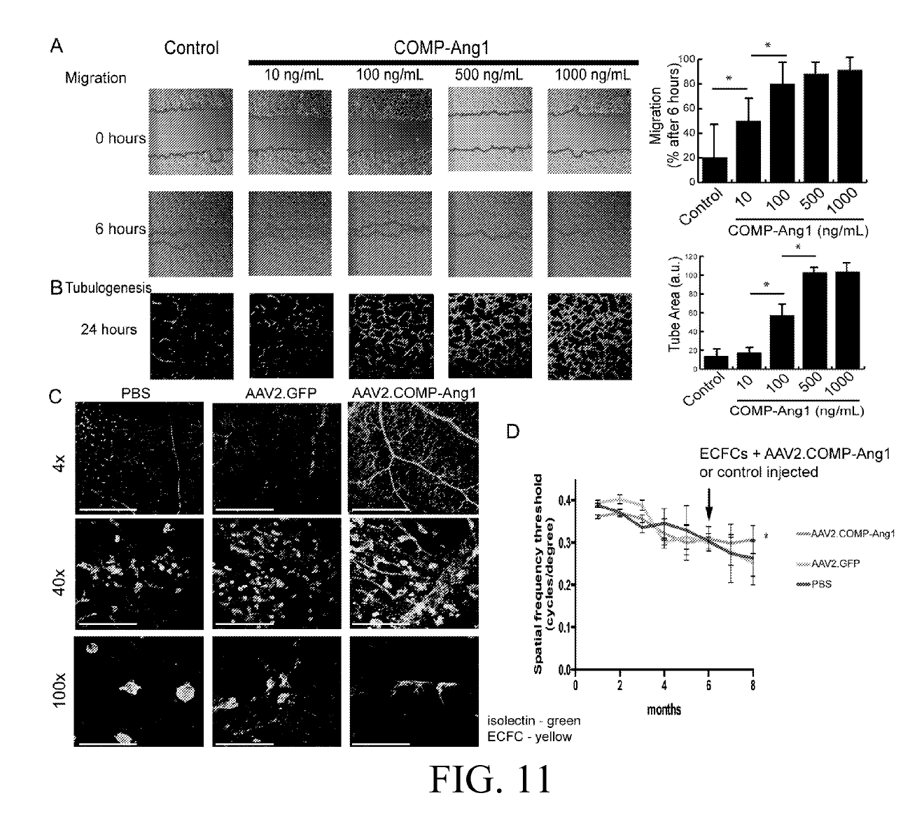

As shown in FIGS. 11A-B, COMP-Ang1 demonstrated a dose-dependent increase in 6-hour migration (2.5-fold increase over control at 10 ng/mL, p<0.01, FIG. 11A) and 24-hour tubulogenesis (4.3 fold over control p<0.01, FIG. 11B) of ECFCs in vitro, as assessed by scratch migration assay and matrigel tube formation assay, respectively. In vivo, aged 26-week-old Ins2Akita mice treated with AAV2.COMP-Ang1 had increased 72-hour ECFC vessel integration on confocal microscopy (FIG. 11C) and 2-month visual response with OKT (AAV2.COMP-Ang1=0.307, AAV2.AcGFP=0.251, PBS=0.263 cycles/degree; p<0.01; FIG. 11D).

These results show that COMP-Ang1 boosts the capacity of ECFCs to rebuild vessels and counteract vision loss in DR.

### Example 9: COMP-Ang1 Regenerates Neuronal Function by Reprogramming Müller Glial Cells

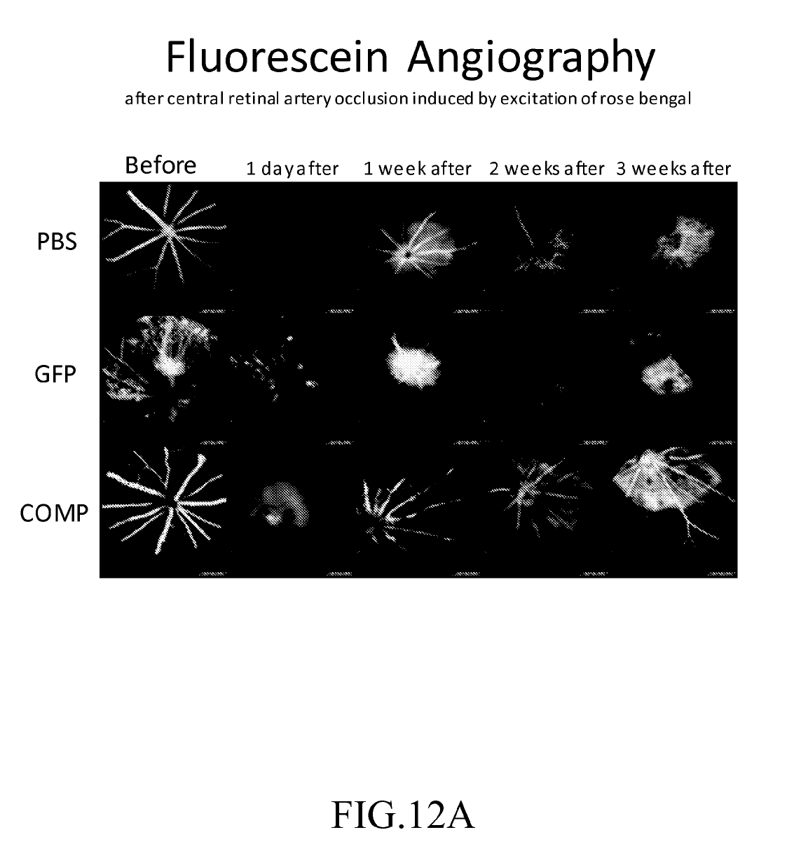

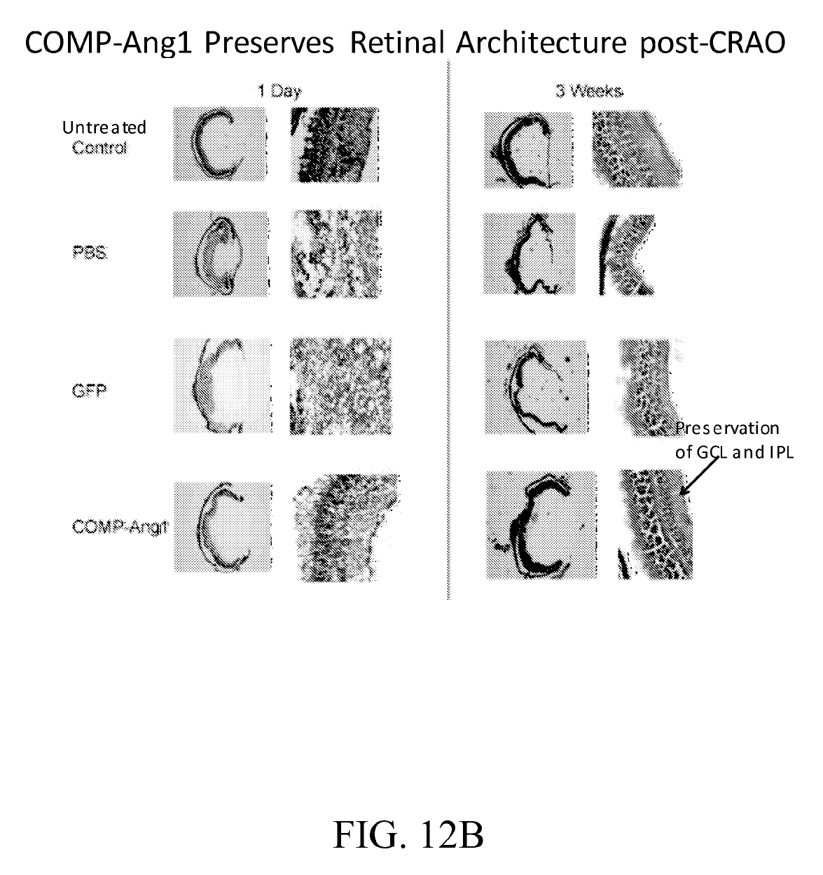

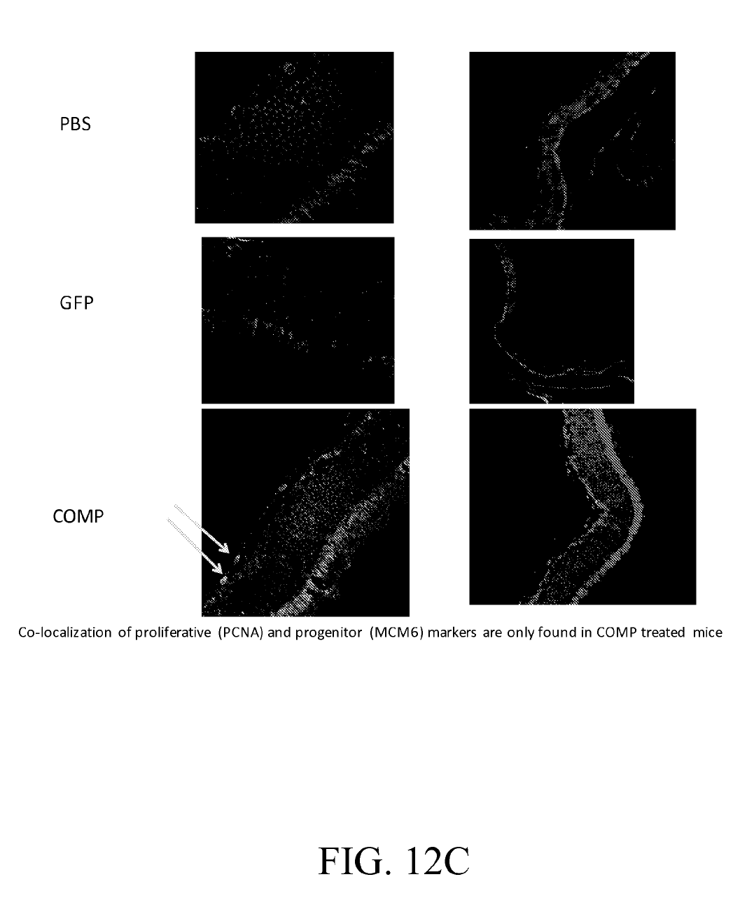

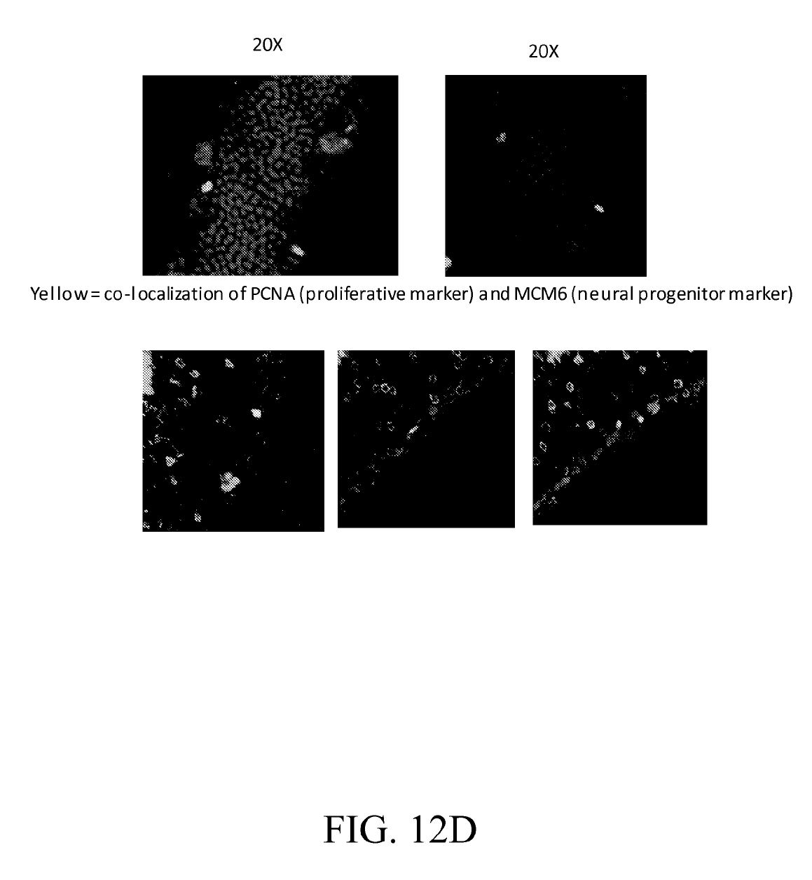

Mice were pretreated with a single injection of AAV2.COMP-Ang1, COMP-AcGFP, or PBS. One month after treatment mice underwent Rose Bengal tail vein injection followed by laser excitation to induce central retinal artery occlusion (CRAO). Occlusion was confirmed by the absence of fluorescein in the retinal vessels using fluorescein angiography, as shown in FIG. 12A. Reperfusion occurred somewhere between 24-48 hours. Leakage of fluorescein was observed for a period of three weeks after occlusion. As can be seen from FIGS. 12A-B, mice treated with COMP-Ang1 had increased vascular restoration and greater preservation of retinal architecture after occlusion. Furthermore, as can be seen by FIGS. 12C-D, proliferative marker PCNA and neurogenesis marker MCM6 were observed in mice treated with COMP-Ang1. This indicates that COMP-Ang1 can initiate neuronal regeneration in situ.

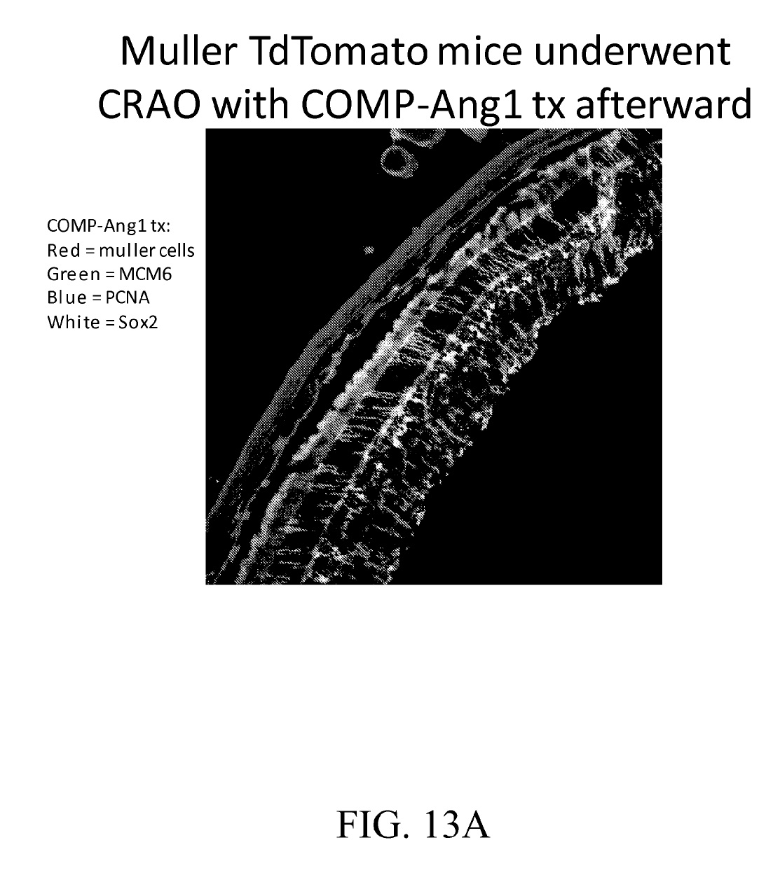

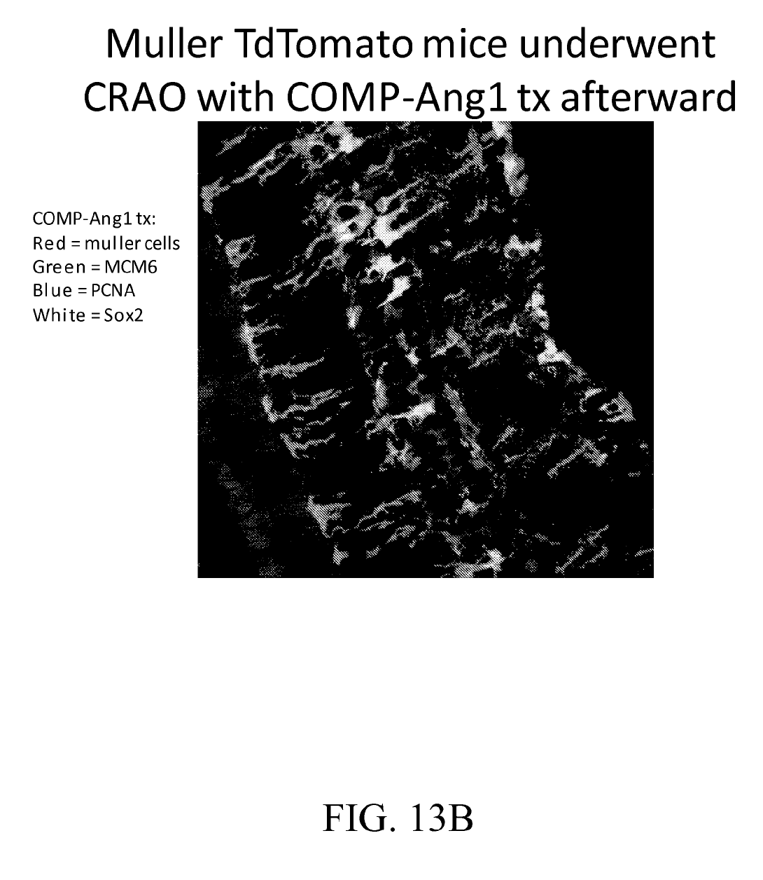

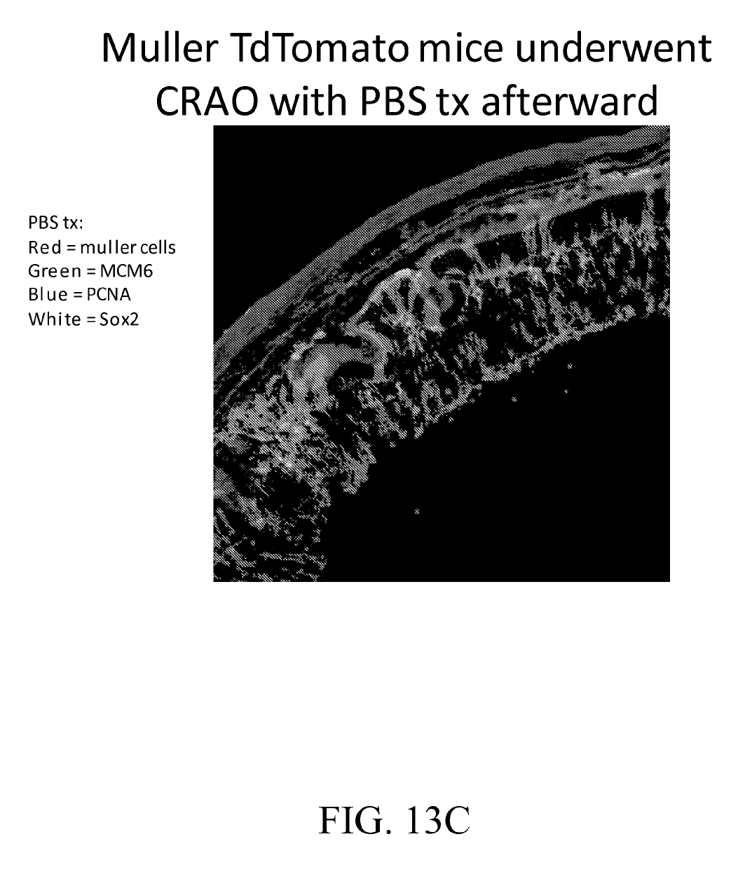

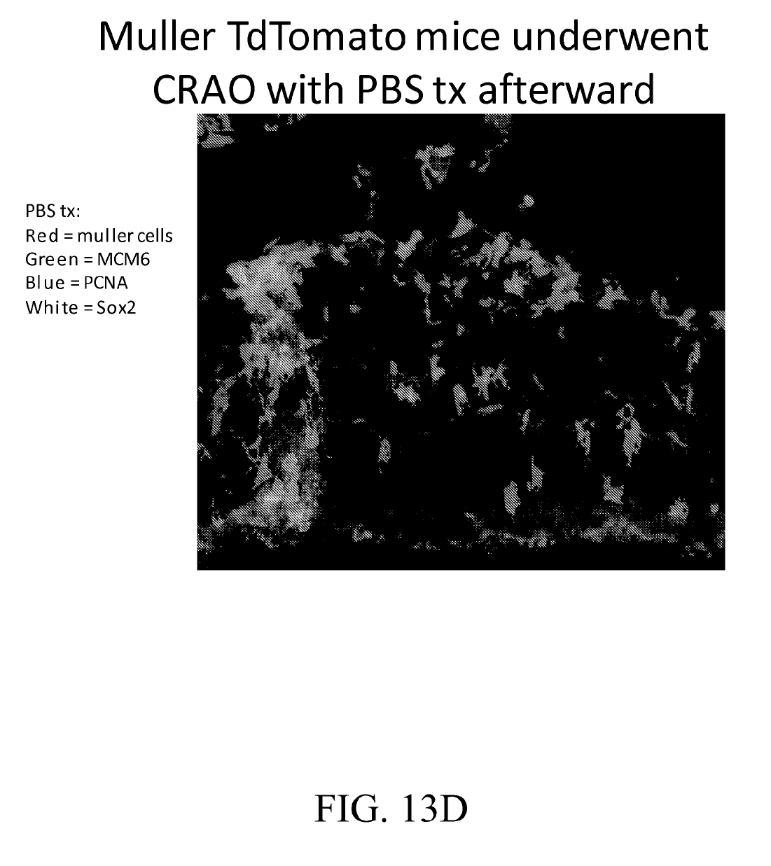

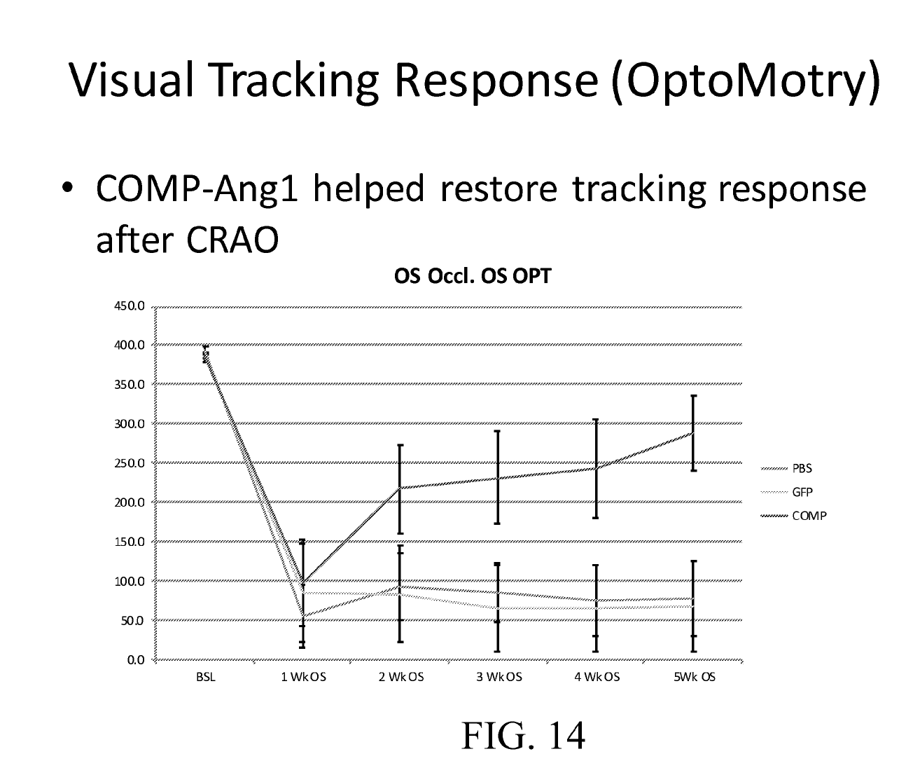

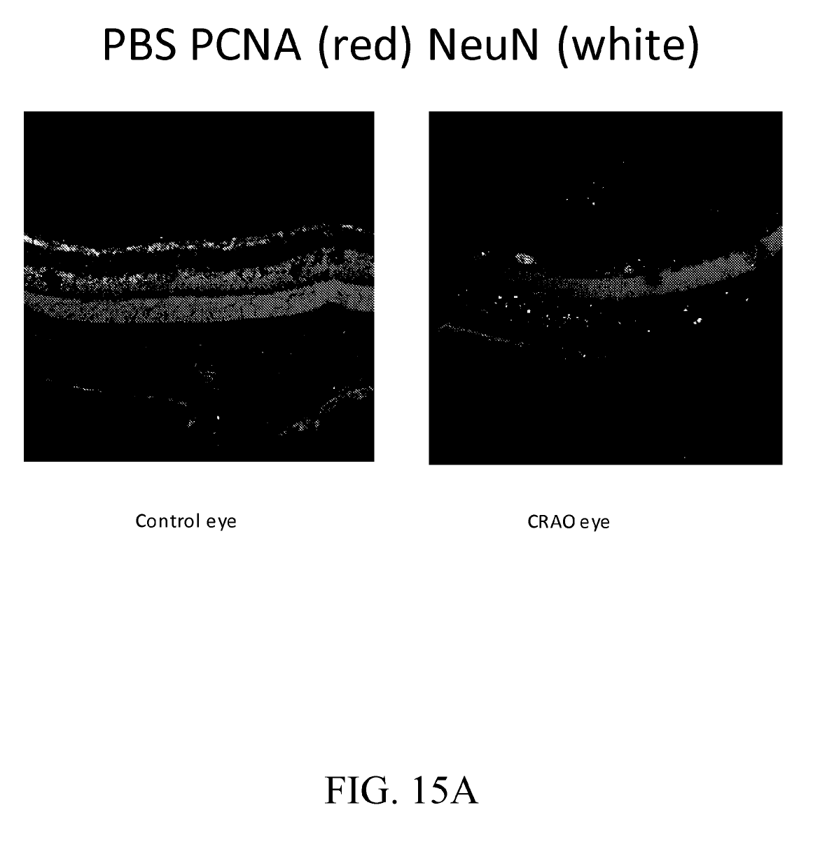

Mice that express TdTomato, a red fluorescent protein, in Muller cells underwent CRAO and were subsequently treated with either COMP-Ang1 protein, GFP, or PBS at both 0 days and 7 days post-treatment. Cross sections of retina samples taken from each treatment group were stained for Muller cells, proliferative marker PCNA, and progenitor markers MCM6 and SOX2. As can be seen from FIGS. 13A-D, COMP-Ang1 treated mice exhibit a significant decrease in Muller cells and a significant increase in progenitor markers as compared to the control mice. Additionally, the visual tracking response of the various treated mice was also monitored using OptoMotry. As can be seen in FIG. 14, COMP-Ang1 also helped restore the tracking response of mice after CRAO.

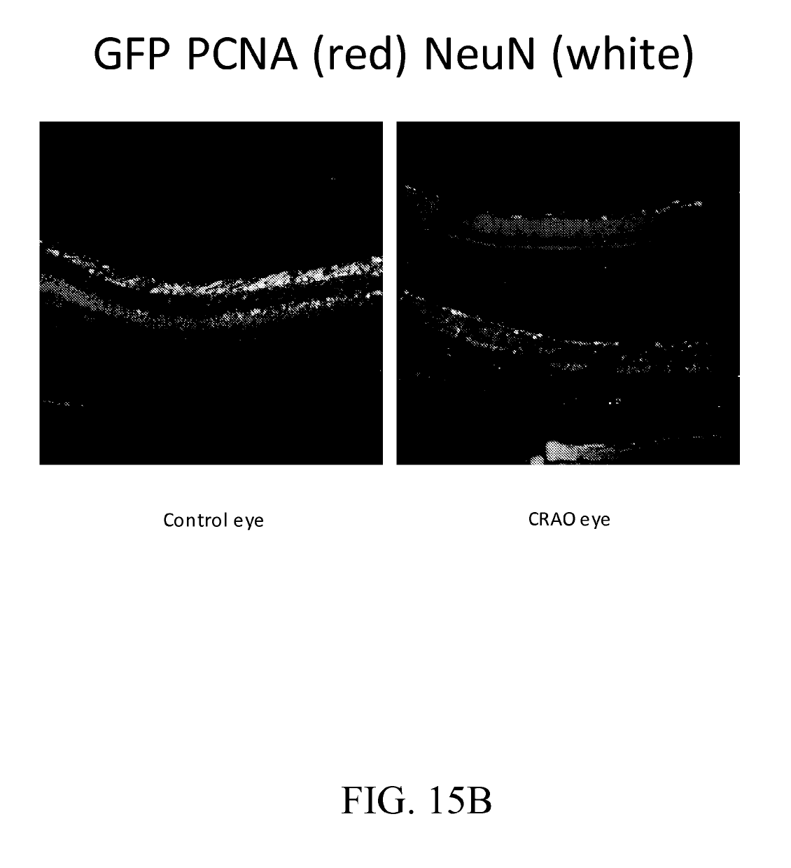

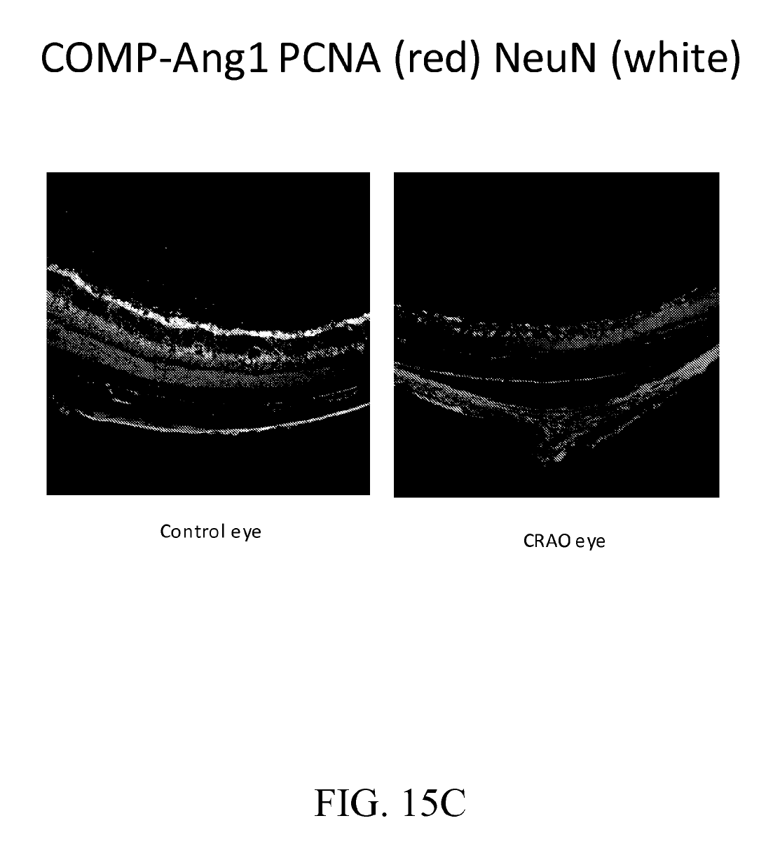

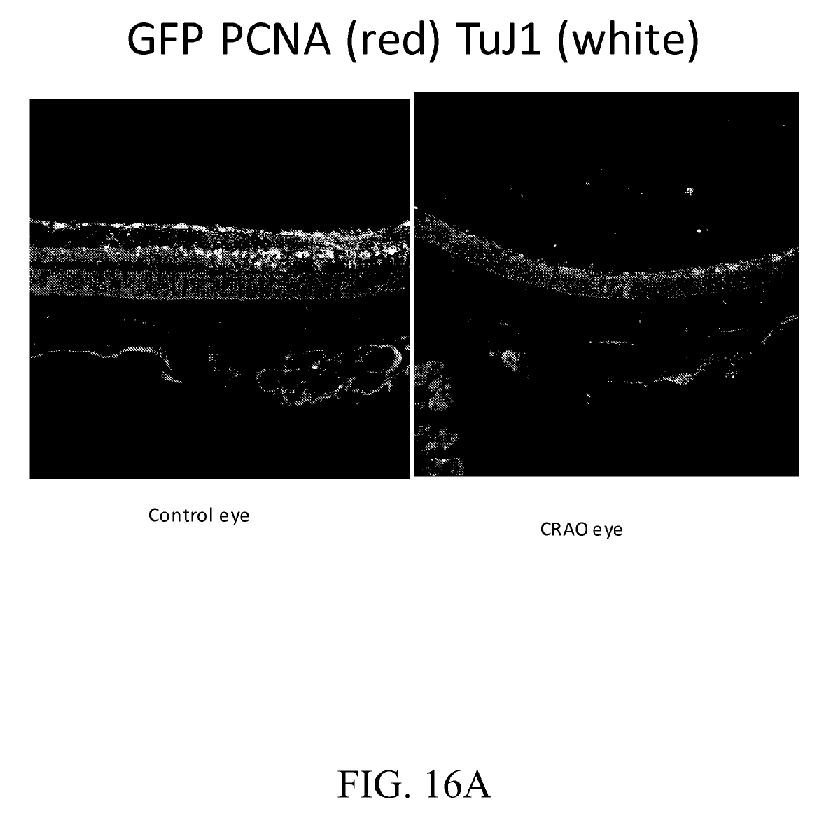

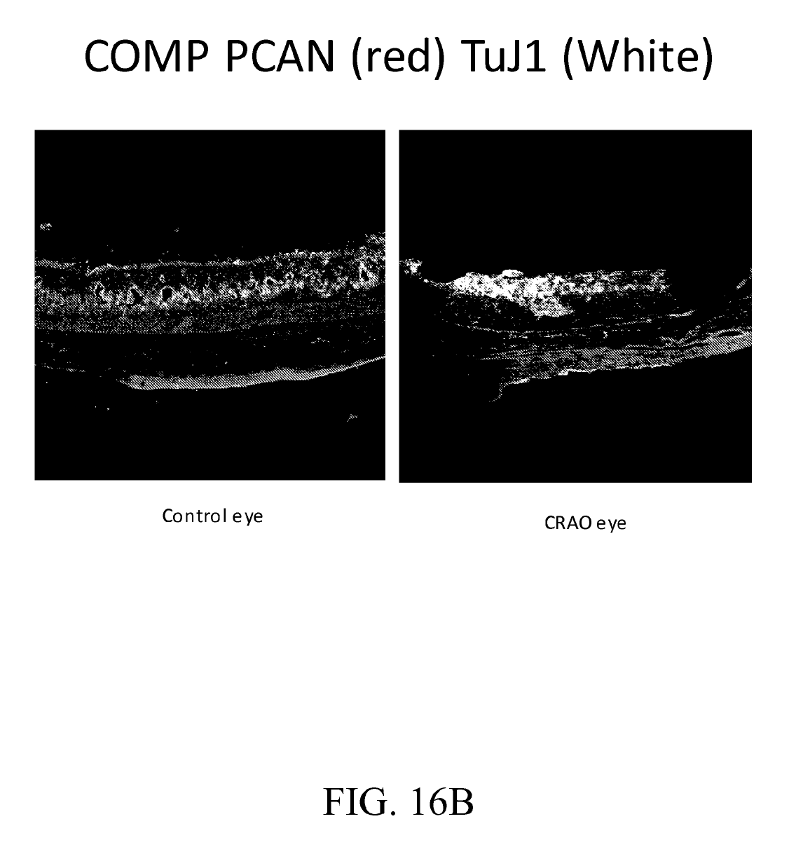

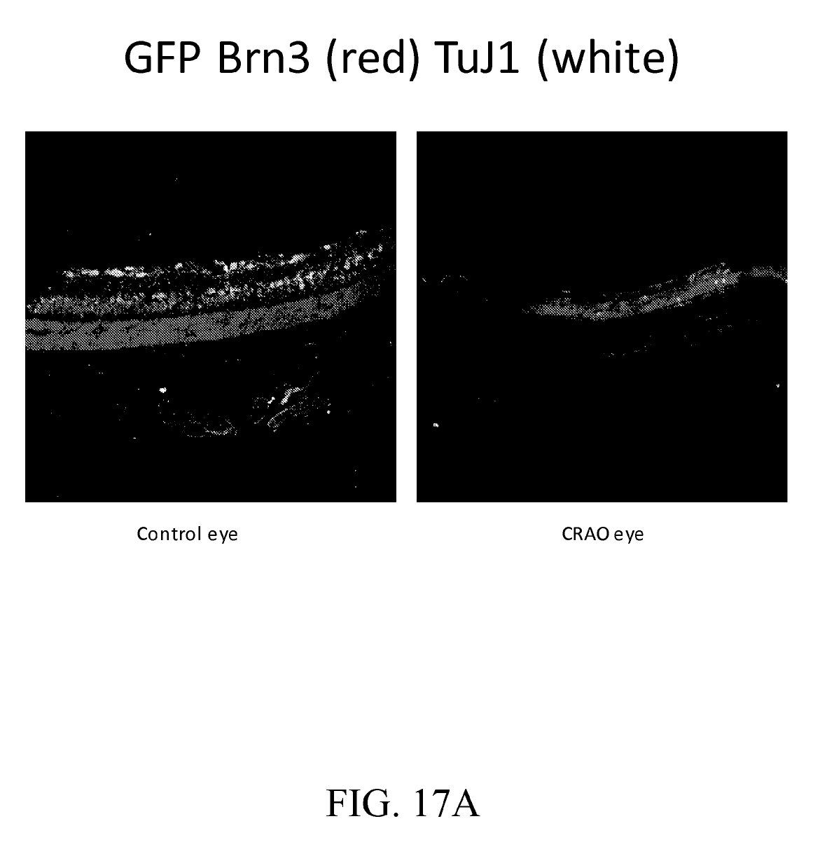

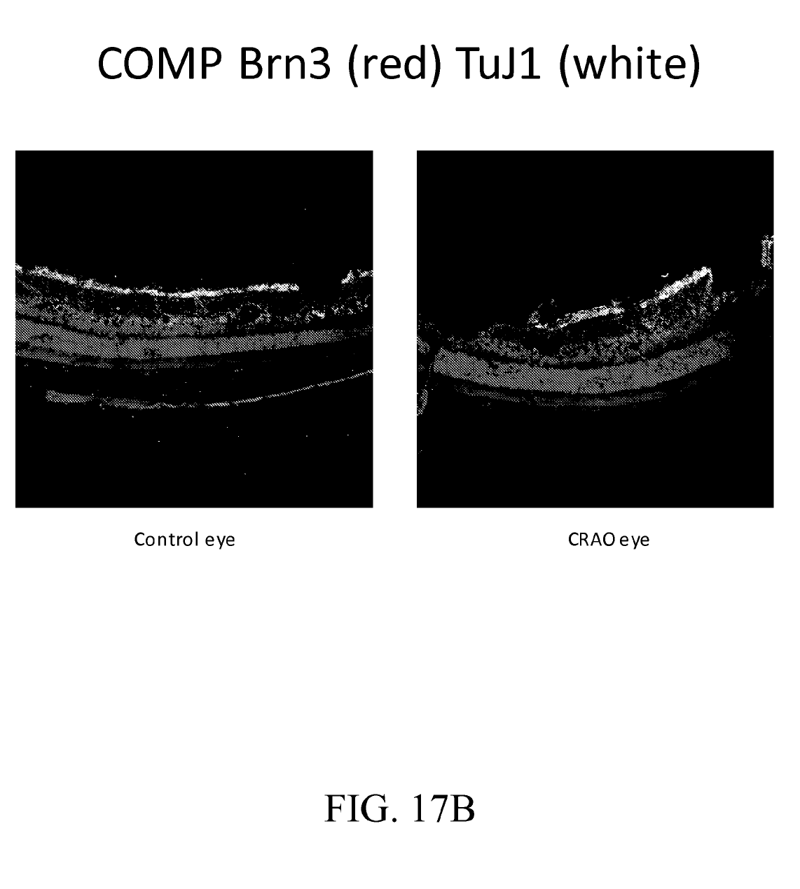

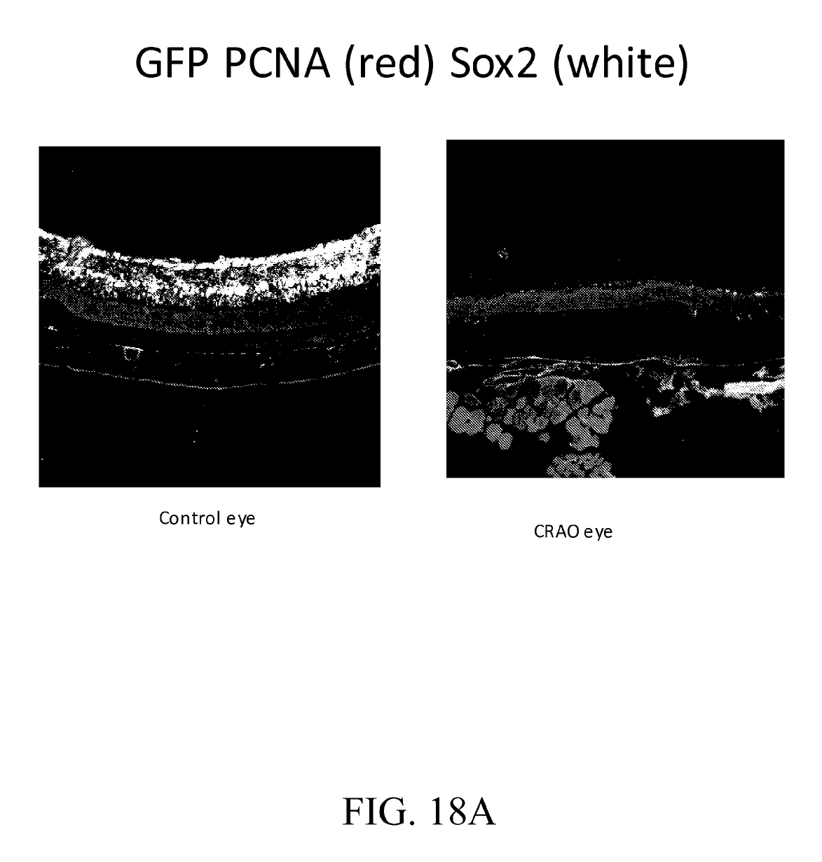

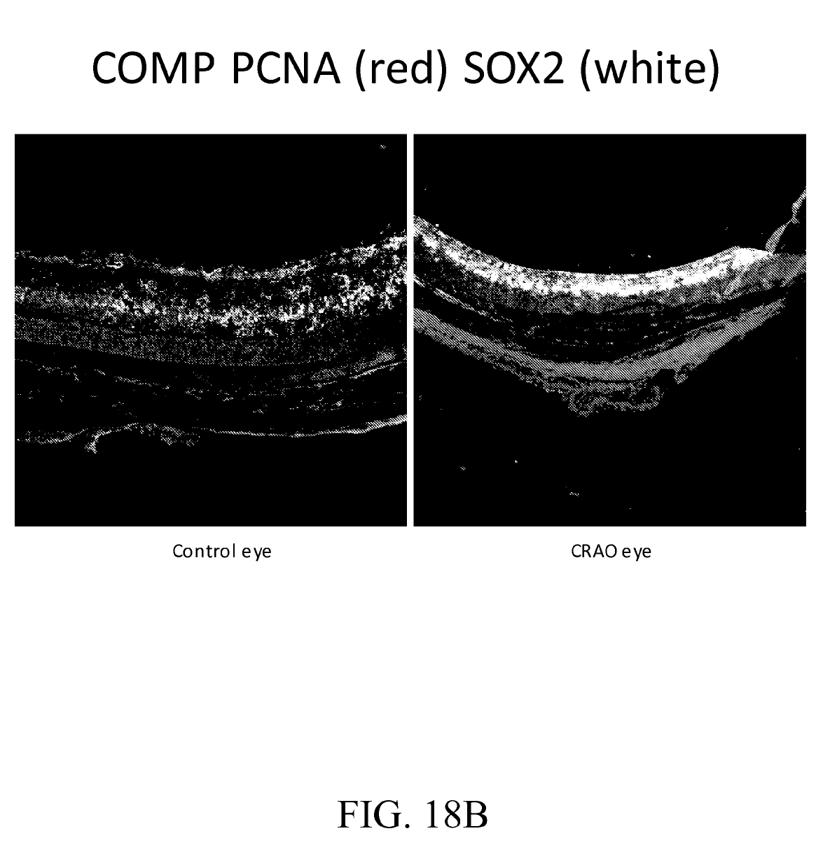

The combination of improved tracking response, decreased Muller cells, and increased progenitor markers is evidence that COMP-Ang1 can reprogram Muller cells into neural cells to restore visual function. This was further evaluated by monitoring TuJ1 and NeuN as neuronal labels and Brn3 as a promoter of ganglion cell differentiation. As shown in FIGS. 15A-C, increased PCNA and NeuN proliferation is seen in both the CRAO and control eye of mice treated with COMP-Ang1 compared to PBS and GFP treated mice. This indicates a greater presence of viable neural cells in the retina of COMP-Ang1 treated mice. Further, as can be seen in FIGS. 16A-B, increased PCNA and TuJ1 proliferation is also seen in both the CRAO and control eye of mice treated with COMP-Ang1 compared to GFP treated mice. This is also evidence of higher concentrations and proliferation of neural cells in COMP-Ang1 treated mice. Moreover, Brn3 was monitored to determine if there was increased promotion of differentiation to ganglion cells in the retina. As can be seen in FIGS. 17A-B, there is an increase in TuJ1 and Brn3 in COMP-Ang1 treated mice compared to GFP treated mice. This indicates that there is increased promotion of ganglion cell differentiation and neural cell concentration in the retina of mice treated with COMP-Ang1. Further still, as can be seen in FIGS. 18A-B, PCNA and SOX2 increased in the retina of mice treated with COMP-Ang1 in both control and CRAO eyes as compared to GFP treated mice. This increase in the progenitor marker SOX2 indicates a greater presence of progenitor cells in the retina of mice treated with COMP-Ang1. The results shown in FIGS. 15-18, accompanied with a decrease in Muller cells and increased visual tracking of mice treated with COMP-Ang1, indicate that the Muller cells are being dedifferentiated to a progenitor phenotype and that the progenitor cells are subsequently differentiating into neuronal cells to restore neuronal function in the retina.

These examples establish the salutary effects of COMP-Ang1 in ameliorating pivotal pathogenic events in the trajectory toward DME, CRAO, and other related conditions through normalization of the neurovasculature. Structural and functional indices of neurovascular restoration by COMP-Ang1 to a state more consistent with a homeostatic disease-free phenotype included at least endothelial and capillary density; vessel permeability; VEGF-A, phospho-Src, VE-cadherin, and phospho-Akt levels; leukocyte-endothelial interaction and retinal hypoxia; neuroretinal thickness, GC-IPL cellularity, and peripheral RGC density; and most importantly, vision. Furthermore, COMP-Ang1 augmented the effectiveness of ECFCs in regenerating vessels and stabilizing vision in advanced DR.

In, sum these results show that COMP-Ang1 is a safe and effective replacement for endogenous Ang1, which can adequately compensate for deficient Ang1 secretion by pericytes. Further, these results indicate that COMP-Ang1 can help preserve retinal architecture, increase re-vascularization of avascular regions after CRAO, increase neurogenesis through Muller cell reprogramming and proliferation, and increase functional visual recovery. These results also indicate that COMP-Ang1 suppresses the pathognomonic features of non-proliferative DR and, in contrast to existing therapies, decreases the non-perfusion and ischemia critical to the genesis of proliferative DR. Because of this, in connection with the long-term duration of action for a single intravitreal injection of AAV2.COMP-Ang1 relative to anti-VEGF agents, a COMP-Ang1 agent, as described herein, can work to manage DR.

While the forgoing examples are illustrative of the specific embodiments in one or more particular applications, it will be apparent to those of ordinary skill in the art that numerous modifications in form, usage and details of implementation can be made without departing from the principles and concepts articulated herein. Accordingly, no limitation is intended except as by the claims set forth below.

### SEQUENCES

SEQ ID 001:
atgaagacgatcatcgccctgagctacatcttctgcctggtattcgccga
ctacaaggacgatgatgacaaggggatcttagacctagccccacagatgc
ttcgagaactccaggagactaatgcggcgctgcaagacgtgagagagctc
ttgcgacagcaggtcaaggagatcaccttcctgaagaatacggtgatgga
atgtgacgcttgcggaggatcccttgtcaatctttgcactaaagaaggtg
ttttactaaagggaggaaaaagagaggaagagaaaccatttagagactgt
gcagatgtatatcaagctggttttaataaaagtggaatctacactattta
tattaataatatgccagaacccaaaaaggtgttttgcaatatggatgtca
atgggggaggttggactgtaatacaacatcgtgaagatggaagtctagat
ttccaaagaggctggaaggaatataaaatgggttttggaaatccctccgg
tgaatattggctggggaatgagtttatttttgccattaccagtcagaggc
agtacatgctaagaattgagttaatggactgggaagggaaccgagcctat
tcacagtatgacagattccacataggaaatgaaaagcaaaactataggtt
gtatttaaaaggtcacactgggacagcaggaaaacagagcagcctgatct
tacacggtgctgatttcagcactaaagatgctgataatgacaactgtatg
tgcaaatgtgccctcatgttaacaggaggatggtggtttgatgcttgtgg
cccctccaatctaaacggaatgttctatactgcggggcaaaaccatggaa
aactgaatgggataaagtggcactacttcaaagggcccagttactcctta
cgttccacaactatgatgattcgacctttagatttttga

What is claimed is:

1. A method of treating a disease or disorder of the eye, comprising administering a therapeutically effective amount of a cartilage oligo matrix protein-Angiopoietin 1 (COMP-Ang1) agent to an eye of a subject during a treatment period.

2. The method of claim 1, wherein treating includes reprogramming a Müller cell to function as a neural cell.

3. The method of claim 1, wherein the disease or disorder of the eye is a member selected from the group consisting of neurovascular dysfunction, neuronal dysfunction, vascular hyperpermeability, retinal ischemia, retinal hypoxia, retinal hypoglycemia, retinal hyperglycemia, retinal stroke, central retinal artery occlusion (CRAO), central retinal vein occlusion, diabetic retinopathy, diabetic macular edema, pericyte dropout, endothelial cell dropout, capillary dropout, decreased blood-retinal barrier (BRB) integrity, leukocyte adhesion, and inflammation.

4. The method of claim 1, further comprising administering a therapeutically effective amount of progenitor cells to an eye of the subject.

5. The method of claim 4, wherein the therapeutically effective amount of progenitor cells is from about 5,000 cells to about 60,000,000 cells.

6. The method of claim 1, wherein administering includes injection.

7. The method of claim 1, wherein the COMP-Ang1 agent is a COMP-Ang1 protein or a homologue thereof.

8. The method of claim 7, wherein the therapeutically effective amount is from about 0.001 mg to about 5 mg of COMP-Ang1 protein or a homologue thereof.

9. The method of claim 1, wherein the COMP-Ang1 agent is an expression vector that induces expression of a COMP-Ang1 protein or a homologue thereof.

10. The method of claim 9, wherein the expression vector is a member selected from the group consisting of a lentivirus, an adenovirus, a cytomegalovirus, an adeno-associated virus (AAV), and combinations thereof.

11. The method of claim 10, wherein the viral particle is an AAV which is a member selected from the group consisting of AAV2, AAV9, AAV10, and combinations thereof.

12. The method of claim 11, wherein the viral particle is an AAV2.

13. The method of claim 9, wherein the therapeutically effective amount is from about 1×10⁹ to about 1×10¹¹ vector units.

14. The method of claim 1, wherein the therapeutically effective amount is from about 0.01 ml to about 1 ml.

15. The method of claim 1, wherein the subject is a human subject.

16. The method of claim 1, wherein the subject is a veterinary subject.

17. The method of claim 1, wherein the treatment period occurs during or after an ischemic or hypoxic event.

18. The method of claim 17, wherein the treatment period is initiated within 72 hours of the onset of the ischemic or hypoxic event.

19. The method of claim 1, wherein the treatment period occurs prior to an ischemic or hypoxic event.

20. The method of claim 1, wherein the treatment period is a period of about 3 weeks.

21. The method of claim 20, wherein a treatment regimen includes administering a therapeutically effective amount of the COMP-Ang1 agent up to three times during the treatment period.

22. The method of claim 1, wherein the treatment period is about 6 months.

23. The method of claim 22, wherein a single administration of the COMP-Ang1 agent is effective for at least 6 months.

24. A therapeutic construct for treating an ocular disease or disorder, comprising:
a vector backbone contained in a viral particle, the vector backbone including a sequence that is at least 80% homologous with SEQ ID 001.

25. The therapeutic construct of claim 24, wherein the viral particle is a member selected from the group consisting of a lentivirus, a cytomegalovirus, an adenovirus, an AAV, and combinations thereof.

26. The therapeutic construct of claim 25, wherein the viral particle is AAV2.

27. A composition for treating a disease or disorder of the eye, comprising:
a therapeutically effective amount of a COMP-Ang1 agent; and
a pharmaceutically acceptable carrier.

28. The composition of claim 27, wherein the COMP-Ang1 agent is a COMP-Ang1 protein or homologue thereof.

29. The composition of claim 28, wherein the therapeutically effective amount is from about 0.001 mg to about 5 mg of COMP-Ang1 protein or a homologue thereof.

30. The composition of claim 27, wherein the COMP-Ang1 agent is an expression vector that is configured to express a COMP-Ang1 protein or a homologue thereof.

31. The composition of claim 30, wherein the expression vector is a member selected from the group consisting of a lentivirus, an adenovirus, a cytomegalovirus, an adeno-associated virus (AAV), and combinations thereof.

32. The composition of claim 31, wherein the viral particle is an AAV which is a member selected from the group consisting of AAV2, AAV9, AAV10, and combinations thereof.

33. The composition of claim 32, wherein the viral particle is an AAV2.

34. The composition of claim 30, wherein the therapeutically effective amount is from about 1×10⁹ to about 1×10¹¹ vector units.

35. The composition of claim 27, wherein the therapeutically effective amount is from about 0.01 ml to about 10 ml.

36. The composition of claim 27, wherein the therapeutically effective amount is from about 5 ml to about 100 ml.

37. The composition of claim 27, wherein the pharmaceutically acceptable carrier includes at least one of a solubilizing agent, a tonicity agent, a pH adjuster, a stabilizing agent, a disaggregation agent, and combinations thereof.

38. The composition of claim 27, wherein the pharmaceutically acceptable carrier includes at least one of Ringer's lactate, normal saline, phosphate-buffered saline (PBS), a balanced salt solution, albumin, and a copolymer of ethylene oxide and propylene oxide.

39. The composition of claim 27, wherein the pharmaceutically acceptable carrier comprises Ringer's lactate and normal saline.

40. The composition of claim 27, wherein the pharmaceutically acceptable carrier comprises albumin, PBS, balanced salt solution, and a copolymer of ethylene oxide and propylene oxide.

41. The composition of claim 27, wherein the pH is from about 6.5 to about 7.8.

42. The composition of claim 27, wherein the tonicity is from about 277 to about 310 mOsm/L.

43. The composition of claim 27, further comprising a therapeutically effective amount of progenitor cells.

44. The composition of claim 43, wherein the therapeutically effective amount of progenitor cells is from about 5,000 cells to about 60,000,000 cells.

45. A system for treating a disease or disorder in an eye of a subject, comprising:
a composition, comprising:
a therapeutically effective amount of a COMP-Ang1 agent; and
a pharmaceutically acceptable carrier; and
a container configured to house and store the composition prior to administration thereof to a subject.

46. The system of claim 45, the composition further comprising a therapeutically effective amount of progenitor cells.

47. The system of claim 45, wherein the composition is a pre-mixed composition that is ready to administer without further dilution.

48. The system of claim 45, wherein the container is an amber-colored container.

49. The system of claim 45, wherein the container is made a material selected from the group consisting of glass, polyethylene, polypropylene, and combinations thereof.


SEQUENCE LISTING (1 sequence)

SEQ ID NO: 1 - DNA, 939 bp, Artificial Sequence
  homo sapiens
atgaagacgatcatcgccctgagctacatcttctgcctggtattcgccgactacaaggacgatgatgacaaggggatcttagacctagccccacagatgcttcgagaactccaggagactaatgcggcgctgcaagacgtgagagagctcttgcgacagcaggtcaaggagatcaccttcctgaagaatacggtgatggaatgtgacgcttgcggaggatcccttgtcaatctttgcactaaagaaggtgttttactaaagggaggaaaaagagaggaagagaaaccatttagagactgtgcagatgtatatcaagctggttttaataaaagtggaatctacactatttatattaataatatgccagaacccaaaaaggtgttttgcaatatggatgtcaatgggggaggttggactgtaatacaacatcgtgaagatggaagtctagatttccaaagaggctggaaggaatataaaatgggttttggaaatccctccggtgaatattggctggggaatgagtttatttttgccattaccagtcagaggcagtacatgctaagaattgagttaatggactgggaagggaaccgagcctattcacagtatgacagattccacataggaaatgaaaagcaaaactataggttgtatttaaaaggtcacactgggacagcaggaaaacagagcagcctgatcttacacggtgctgatttcagcactaaagatgctgataatgacaactgtatgtgcaaatgtgccctcatgttaacaggaggatggtggtttgatgcttgtggcccctccaatctaaacggaatgttctatactgcggggcaaaaccatggaaaactgaatgggataaagtggcactacttcaaagggcccagttactccttacgttccacaactatgatgattcgacctttagatttttga